(19) United States
(12) Patent Application Publication
(10) Pub. No.: US 2016/0186207 A1
(43) Pub. Date: Jun. 30, 2016

(54) NOVEL METHOD

(71) Applicant: BABRAHAM INSTITUTE, Cambridge, Cambridgeshire (GB)

(72) Inventors: Wolf REIK, Cambridgeshire (GB); Julian PEAT, Cambridgeshire (GB); Timothy HORE, Cambridgeshire (GB); Christel KRUEGER, Cambridgeshire (GB)

(21) Appl. No.: 14/652,742

(22) PCT Filed: Dec. 17, 2013
(86) PCT No.: PCT/GB2013/053317

§ 371 Date: Jun. 16, 2015

(30) Foreign Application Priority Data
Dec. 17, 2012 (GB) 1222693.2

(51) Int. Cl. C12N 15/85; A61K 35/545; C12N 9/02; C12Q 1/68

(52) U.S. Cl. CPC C12N 15/85; C12Q 1/6876; A61K 35/545; C12N 9/0071; C12Y 114/11

(57) ABSTRACT

The invention relates to a method of enhancing the potency of a cell (for example, to a totipotent state), by introducing a TET family gene, derivative or fragment thereof into the cell. The invention also relates to methods and kits for preparing cells with enhanced potency, and uses of said cells.

### FIELD OF THE INVENTION

The invention relates to a method of enhancing the potency of a cell, by introducing a TET family gene, derivative or fragment thereof into the cell. The invention also relates to methods and kits for preparing cells with enhanced potency, and uses of said cells.

### BACKGROUND OF THE INVENTION

It is thought that the use of stem cells could radically change the treatment of human disease. Stem cells are known to have a high level of potency and self-renewal which means that they can be differentiated into multiple cell types. This advantageous property could be used in the generation or repair of organs and tissues.

The isolation of embryonic stem (ES) cells has led to major advances in stem cell technology and research. ES cells are pluripotent, therefore they can be induced to differentiate into multiple cells types which can then be used, for example, in scientific animal models or cell transplantation therapies. However, ES cells have not yet fulfilled their expectations as the solution to most problems currently faced in the treatment of disease. For example, transplantation of ES cells has been shown to face rejection problems in the same manner as current organ transplantation. Furthermore, the use of these cells raises ethical issues in view of the fact that embryos are destroyed during the harvesting of ES cells.

Recently, scientists have developed a way to produce induced pluripotent stem (iPS) cells (as described in WO 2007/069666) which allow a patient's own somatic cells to be de-differentiated into a pluripotent state, thus overcoming the ethical issues associated with ES cells. However, iPS cells and ES cells from humans and other mammals outside the rodent lineage in nearly all cases suffer from a lack of full pluripotency.

Furthermore, as pluripotent cells, ES and iPS cells from any species cannot form tissues of the extra-embryonic lineage and must be injected into a host blastocyst to generate a complete organism.

WO 2010/037001 describes methods of regulating and detecting the cytosine methylation status of DNA using the family of TET proteins in order to reprogram stem cells.

There is therefore a need for a method to produce cells with higher potency, such as totipotent cells, for use in stem cell technology.

### SUMMARY OF THE INVENTION

According to a first aspect of the invention, there is provided a method of enhancing the potency of a cell, wherein said method comprises the step of introducing a TET family gene, derivative or fragment thereof into the cell.

According to a further aspect of the invention, there is provided a method of preparing a cell with enhanced potency which comprises the step of introducing a TET family gene, derivative or fragment thereof into a cell.

According to a further aspect of the invention, there is provided a cell with enhanced potency obtainable by the method as defined herein.

According to a further aspect of the invention, there is provided a nucleic acid comprising a TET3 isoform of SEQ ID NO: 11 or 13.

According to a further aspect of the invention, there is provided a vector comprising the nucleic acid as defined herein.

According to a further aspect of the invention, there is provided the use of the nucleic acid as defined herein, or the vector as defined herein, in a method of enhancing the potency of a cell.

According to a further aspect of the invention, there is provided the cell with enhanced potency as defined herein for use in therapy.

According to a further aspect of the invention, there is provided a kit comprising a vector containing a TET family gene, derivative or fragment thereof and instructions to use said kit in accordance with the method as defined herein.

### BRIEF DESCRIPTION OF THE FIGURES

FIG. 1: Schematic of the 5′ Tet3 locus. Diagram is not to scale. Dotted line represents multiple exons and introns. Arrows indicate positions of qRT-PCR primers used for promoter usage analysis (see Examples Section). Start codons indicated are in-frame with full-length TET3 protein. ‘Cat’=catalytic domain.

FIG. 2: Promoter usage and incorporation of the CXXC-encoding exon. Transcript level is shown relative to the average of reference genes Atp5b and Hspcb. Except for oocyte (which has single values) values shown are the average of two biological replicates with the range shown as error bars. EB: embryoid bodies.

FIG. 3: Expression analysis of candidate genes by qPCR in sorted cells transfected with Tet3 Variant 1. Transcript level is shown relative to the average of reference genes Atp5b and Hspcb. Mut: catalytically inactive mutant.

FIG. 4: Expression analysis of control genes by qPCR in sorted cells transfected with Tet3 Variant 1. Transcript level is shown relative to the average of reference genes Atp5b and Hspcb. Mut: catalytically inactive mutant.

FIG. 5: Expression analysis of candidate genes by qPCR in sorted cells transfected with Tet3 Variant 3. Transcript level is shown relative to the average of reference genes Atp5b and Hspcb. Mut: catalytically inactive mutant.

FIG. 6: Scatterplot of expression levels in sorted cells transfected with Tet3 Variant 1. Each point represents a single gene. Candidate genes examined by qPCR (see Example 4) and several family members are indicated in black, with some example genes labelled with arrows.

FIG. 7: Scatterplot of expression levels in sorted cells transfected with Tet3 Variant 1 catalytic mutant. Each point represents a single gene. Candidate genes examined by qPCR (see Example 4) and several family members are indicated in black, with some example genes labelled with arrows.

FIG. 8: A heatmap showing results of single cell expression data in embryonic stem cells expressing Tet3 Variant 1.

FIG. 9: Graph indicating the proportion of totipotent-like cells in a subpopulation which express TET3.

FIG. 10: Quantitative RT-PCR analysis of TET3 expression. Transcript levels are shown relative to E14 (=1). Values are the average of two independent replicates; error bars indicate the range.

FIG. 11: Phase contrast microscopy of colony morphology after a six-day transdifferentiation assay. Images are representative of the range of colony morphology observed.

FIG. 12: Flow cytometry analysis of CD40 expression after a six-day transdifferentiation assay. After culturing for six days in TS cell media, cells were stained with goat a-CD40 primary antibody (R&D Systems) then anti-goat AlexaFluor 647 secondary antibody (Invitrogen). A: Dot plots showing value of forward scatter width (FSC-W) on the Y-axis and 640 nm fluorescence (i.e. CD40 signal) on the X-axis for individual cells. The threshold for calling CD40 positivity, and the percentage of cells exceeding this level, is indicated. Student's t tests on the total cell population demonstrates a highly significant increase in CD40 positive cells for both TET3-overexpressing cell lines relative to E14 ES cells (p<0.0001 in both cases). B. Quantification of the percentage of cells called as CD40 positive in each cell line.

### DETAILED DESCRIPTION OF THE INVENTION

According to a first aspect of the invention, there is provided a method of enhancing the potency of a cell, wherein said method comprises the step of introducing a TET family gene, derivative or fragment thereof into the cell.

References herein to ‘enhanced potency’ refer to cells which have an increased ability to differentiate into different cell types. Totipotent cells are known to be cells with the highest potency. This is followed by pluripotent, multipotent, oligopotent and then unipotent cells.

In one embodiment, the potency of the cell is enhanced to a pluripotent state, such as a true pluripotent state.

References herein to ‘pluripotent’ refer to cells which have the potential to differentiate into multiple types of cell. These cells are more limited than totipotent cells in that a pluripotent cell alone could not develop into a foetal or adult organism because pluripotent cells cannot differentiate into extra-embryonic cells. Therefore, donor blastocyst cells have to be used in order to generate a complete organism.

As described herein, methods are known in the art to produce iPS cells, however these cells have been shown to lack full pluripotency because they retain an epigenetic memory of their donor somatic cells (Kim et al. (2011) Nature 467, p. 285-290). Therefore, these cells are not considered to be truly pluripotent because they do not have the same ability as natural pluripotent cells to differentiate into multiple cells types.

Therefore, references herein to ‘true pluripotent state’ refer to cells which have the same ability as natural pluripotent cells to differentiate into multiple cells types, i.e. they are fully pluripotent. In particular, truly/completely pluripotent cells can differentiate into any of the three germ layers of the embryo, i.e. the endoderm, mesoderm or ectoderm layers.

In one embodiment, the potency of the cell is enhanced to a totipotent state.

Thus, according to a further aspect of the invention, there is provided a method of reprogramming a cell to a totipotent state, wherein said method comprises the step of introducing a TET family gene, derivative or fragment thereof into the cell.

References herein to ‘totipotent’ refer to cells which have the potential to differentiate into all types of cell, including cells comprising extra-embryonic tissues. Therefore, totipotent cells have the advantage of being able to develop into a complete organism, without needing to use blastocyst cells generated by the host. It will be understood that references to ‘totipotent’ cells, includes ‘totipotent-like’ cells, i.e. cells with a high degree of similarity to totipotent cells, for example a high degree of transcriptional or epigenetic similarity to totipotent cells (see Macfarlan et al. (2012) Nature 487, p. 57-63, which describes a gene expression shift that results in the acquisition of totipotency). Furthermore, references to ‘totipotent’ or ‘totipotent-like’ cells as used herein, refer to cells which have a higher potency than pluripotent cells.

References herein to ‘somatic’ refer to any type of cell that makes up the body of an organism, excluding germ cells and undifferentiated stem cells. Somatic cells therefore include, for example, skin, heart, muscle, bone or blood cells.

As cells differentiate into a particular cell type (e.g. skin, muscle, blood etc.), they lose their ability (or potential) to become a different cell type. It is therefore advantageous to reprogram cells back into a state of pluri- or toti-potency, so that they can be manipulated into a desired cell type.

References herein to ‘reprogramming’ refer to the process by which a cell is converted back into a different state of differentiation. The invention described herein reprograms a cell into a totipotent state, thereby increasing its potency and ability to differentiate into multiple cell types.

Current stem cell technologies rely on the use of ES cells and iPS cells. However, both of these cell types have several disadvantages. For example iPS cells have been shown to retain an epigenetic memory of their donor somatic cells which is not present in natural pluripotent cells (Kim et al. (2011) Nature 467, p. 285-290). Furthermore, ES and iPS cells from humans and other mammals outside the rodent lineage have been shown to not be truly pluripotent. The present invention provides a method of increasing the state of potency of a cell, for example to a totipotent state, thus overcoming these issues associated with human ES and iPS cells.

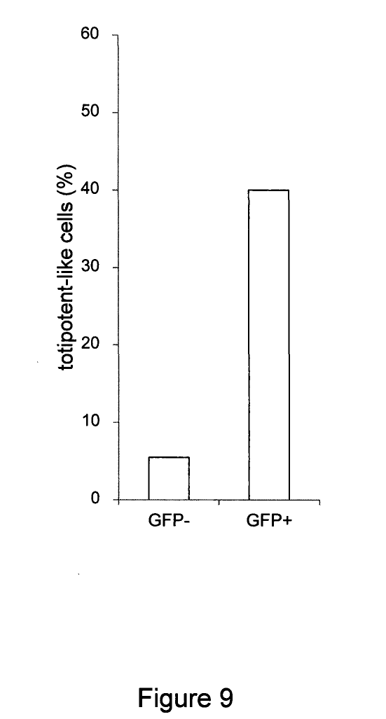

As shown herein, using a TET family gene (e.g. a Tet3 gene) can increase the number of totipotent-like stem cells in a cell culture (see FIG. 9). This subpopulation of totipotent-like stem cells has been shown to have an enhanced potency, as gauged by their ability to transdifferentiate to trophoblast-like cells (see Example 7). Therefore, these cells are able to form extra-embryonic tissues, such as the trophoblast, without the need for donor blastocyst cells.

In one embodiment, the cell is a pluripotent cell. In an alternative embodiment, the cell is a somatic cell.

In one embodiment, the pluripotent cell is from a mammal. In a further embodiment, the mammal is a human.

Pluripotent cells can be obtained from various sources, for example embryonic stem (ES) cells or induced pluripotent stem (iPS) cells, which are commercially available or may be obtained using the methods described in WO 2007/069666. In one embodiment, the pluripotent cell is an induced pluripotent stem (iPS) cell. In an alternative embodiment, the pluripotent cell is an embryonic stem (ES) cell. In a further embodiment, the embryonic stem (ES) cell is an E14 embryonic stem (ES) cell.

The mammalian ten-eleven translocation (TET) family contains three proteins TET1, TET2 and TET3) which all share a high degree of homology between their C-terminal catalytic domains (Iyer et al. (2009) Cell Cycle 8, p. 1698-1710). They have all been shown to convert 5-methylcytosine (5mC) into another form of DNA methylation known as 5-hydroxymethylcytosine (5hmC). The function of 5hmC is still unclear although it is thought to regulate gene expression by removing methyl groups (i.e. through demethylation). The three proteins have fairly different expression profiles and studies so far have shown roles for TET1 in embryonic stem (ES) cells, TET2 in haematopoietic development and cancer, and TET3 in the zygote. In particular, TET3 has been found to be highly expressed in oocytes and fertilized zygotes, as compared to the low levels of TET1 and TET2 (Gu et al. (2011) Nature 477, p. 606-610; Wossidlo et al. (2011) Nature 2, p. 241). The functional differences between the family of three proteins are still unclear.

A major aspect of reprogramming cells to pluripotency is changing their epigenetic landscape, in particular their DNA methylation profile. As part of the demethylation process, 5-methylcytosines are oxidised which is mediated by the catalytic function of TET proteins. Thus, ectopic expression of TET proteins can facilitate reprogramming from somatic cells to pluripotent cells by resetting DNA methylation marks (Costa et al., Nature 495, p. 370-374, WO 2010/037001). Moreover, expression of TET1 and TET2 is high in pluripotent cells, as are levels of oxidised 5-methylcytosine residues in DNA.

However, the present inventors have made the surprising discovery that expression of TET proteins (e.g. TET3) can enhance the potency of cells towards a totipotent state. This enhancement of potency is also likely to affect somatic cells during reprogramming. Unexpectedly, this enhancement of potency is not dependent on the catalytic function of the TET protein and is therefore not linked to DNA demethylation. Thus, expansion of potency towards totipotency is a previously undescribed function of TET proteins.

References herein to a ‘TET family gene’ refer to genes encoding one of the three proteins of the ten-eleven translocation (TET) family: TET1, TET2 or TET3. Such references include genes having at least 70%, at least 75%, at least 80%, at least 85%, at least 90%, at least 95%, at least 98%, at least 99%, or more, sequence identity to TET1, TET2, or TET3, in particular human TET1, TET2, or TET3.

The invention also includes methods of using fragments of a TET family gene. Such fragments usually encode proteins of at least 5 amino acids in length. In preferred embodiments, they may encode proteins of 6 to 10, 11 to 15, 16 to 25, 26 to 50, 51 to 75, 76 to 100 or 101 to 250 or 250 to 500, 500 to 1000, 1000 to 1500 or 1500 to 2000 amino acids. Fragments may include sequences with one or more amino acids removed, for example, C-terminus truncated proteins. Fragments may also include nucleic acids which encode proteins without a particular domain, for example fragments where the CXXC (DNA-binding) domain, or catalytic domain is absent.

References to a ‘TET family derivative’ refer to nucleic acids which encode protein variants of the TET family proteins, which have a different nucleic acid sequence to the original gene, but produce a protein which is considered to be equivalent in shape, structure and/or function. Changes which result in production of chemically similar amino acid sequences are included within the scope of the invention. Variants of the polypeptides of the invention may occur naturally, for example, by mutation, or may be made, for example, with polypeptide engineering techniques such as site directed mutagenesis, which are well known in the art for substitution of amino acids.

Changes in the nucleic acid sequence of the TET family gene of interest can result in conservative changes or substitutions in the amino acid sequence. Therefore, the invention includes polypeptides having conservative changes or substitutions. The invention includes sequences where conservative substitutions are made that do not compromise the activity of the TET family protein of interest.

The inventors of the present invention have made the surprising discovery that introduction of members of the TET family of enzymes (in particular TET3) cause an increase in potency of the cell, for example to a totipotent state.

In one embodiment, the TET family gene, derivative or fragment thereof, is TET2 or TET3 gene, derivative or fragment thereof. In a further embodiment, the TET family gene, derivative or fragment thereof, is a TET3 gene, derivative or fragment thereof. In a yet further embodiment, the TET family gene, derivative or fragment thereof, is TET3, in particular human TET3.

In one embodiment, the TET family gene, derivative or fragment thereof, is a TET3 isoform selected from SEQ ID NOs: 11, 12 or 13, in particular SEQ ID NO: 11 or 13. In one embodiment, the TET family gene, derivative or fragment thereof, is a TET3 isoform of SEQ ID NO: 11 (Tet3 Variant 1). In an alternative embodiment, the TET family gene, derivative or fragment thereof, is a TET3 isoform of SEQ ID NO: 13 (Tet3 Variant 3).

The TET family gene, derivative or fragment thereof may comprise at least 70%, at least 75%, at least 80%, at least 85%, at least 90%, at least 95%, at least 98%, at least 99%, or more sequence identity to SEQ ID NO: 11 or 13.

In one embodiment, the introducing step comprises transfecting the cell with a vector containing the TET family gene, derivative or fragment thereof. In a further embodiment, the vector is a transposon vector.

Vectors are used to introduce a target sequence acid into a host cell using techniques well known in the art (for example, see Example 3 as described herein). A vector may also contain various regulatory sequences that control the transcription and translation of the target sequence. Examples of vectors include: viral vectors, transposon vectors, plasmid vectors or cosmid vectors.

Possible vectors for use in the present invention are commercially available from various suppliers, for example from Invitrogen, Inc. (e.g. Gateway® Cloning Technology), Amersham Biosciences, Inc. and Promega, Inc.

Transposon vectors utilise mobile genetic elements known as transposons to move target sequences to and from vectors and chromosomes using a “cut and paste” mechanism. Examples of transposon vectors include PiggyBac vectors (System Biosciences) or EZ-Tn5™ Transposon Construction vectors (Illumina, Inc.).

Viral vectors consist of DNA or RNA inside a genetically-engineered virus. Viral vectors may be used to integrate the target sequence into the host cell genome (i.e. integrating viral vectors). Examples of viral vectors include adenoviral vectors, adenoviral-associated vectors, retroviral vectors or lentiviral vectors (e.g. HIV).

Plasmid vectors consist of generally circular, double-stranded DNA. Plasmid is vectors, like most engineered vectors, have a multiple cloning site (MCS), which is a short region containing several commonly used restriction sites which allows DNA fragments of interested to be easily inserted.

References herein to ‘transfection’ refer to the process by which the vector is introduced into the host cell so that the target sequence can be expressed. Methods of transfecting the host cell with the vector include electroporation, sonoporation or optical transfection, which are methods well known in the art.

It should be noted that other types of transfection may be envisaged for the present invention, for example particle-based methods which use nanotechnology. In one embodiment, the TET family gene, derivative or fragment thereof is attached to a nanoparticle. The nanoparticle can then be used to transfect the cell, e.g. through use of a ‘gene gun’ (or ‘biolistic particle delivery system’) which delivers the nanoparticle directly into the nucleus of the cell.

Once the vector has been transfected into the cell, the cell may be induced to express the target sequence. Certain vectors, for example transposon vectors, may use excision-based methods in order to excise the target sequence from the vector and deliver it into the host cell's genome where it is expressed. Examples of excision-based methods include piggyBAC technology, Sleeping Beauty (SB) transposons, LINE1 (L1) retrotransposons or CreloxP recombination.

Excision-based methods may use transposons in order to deliver the target sequence into the host genome. The piggyBAC transposon has the particular advantage of being able to excise the target sequence without leaving any exogenous DNA remnants which could affect the reprogramming process.

According to a further aspect of the invention, there is provided a method of preparing a cell with enhanced potency which comprises the step of introducing a TET family gene, derivative or fragment thereof into a cell.

According to a further aspect of the invention, there is provided a method of preparing a reprogrammed totipotent cell which comprises the step of introducing a TET family gene, derivative or fragment thereof into a cell.

In one embodiment, the cell is a pluripotent cell.

In an alternative embodiment, the cell is a somatic cell. In a further embodiment, when the cell is a somatic cell, the method additionally comprises the step of introducing a Oct3/4 gene, a Sox2 gene, a Klf4 gene and a c-Myc gene into the somatic cell.

The method defined herein may be used to induce a somatic cell (for example, a somatic cell obtained from a patient) into a pluripotent or a totipotent state. It will be understood that this may be achieved in one step, or by inducing the somatic cell into a pluripotent state and then a totipotent state. For example, TET (e.g. TET3) overexpression in concert with existing overexpression systems, such as Yamanaka factors, may allow derivation of totipotent cells from somatic cells in essentially one experimental step.

There are methods widely available in the art for inducing somatic cells into a pluripotent state, for example by introducing Yamanaka factors (i.e. Oct3/4, Sox2, Klf4 and c-Myc genes, as described in WO 2007/069666). These factors may be introduced using a vector containing the four factors, such as Plasmid 20959 (PB-TET-MKOS) available from www.addgene.org. Therefore, a somatic cell may be reprogrammed into a totipotent state by co-transfecting a somatic cell with a vector containing the TET family gene, derivative or fragment thereof and a vector containing the Oct3/4, Sox2, Klf4 and c-Myc genes, using the methods as described herein.

References herein to ‘reprogrammed totipotent cell’ refer to a cell which has been induced into a totipotent state by increasing its potency via the introduction of a TET family gene, derivative or fragment thereof.

Methods of introducing nucleic acid sequences of interest into host cells are well known in the art. For example, one basic protocol involves the steps of:

a) Amplification of the nucleic acid target sequence (e.g. a TET family gene, derivative or fragment thereof);

b) Recombination of the target sequence into a vector (e.g. a viral vector);

c) Identification of a successful recombinant using a selectable marker (e.g. green fluorescent protein);

d) Transfection of the recombinant vector into a host cell (e.g. a pluripotent cell or somatic cell);

e) Integration of the target sequence into the host cell genome (e.g. using piggyBAC technology);

f) Identification of successful integration using a selectable marker (e.g. puromycin);

g) Inducing expression of the target sequence (e.g. using doxycycline); and

h) Selection of reprogrammed totipotent cells which successfully express the target sequence (e.g. using flow cytometry).

In one embodiment, the method further comprises the step of culturing the cell after introduction of the TET family gene, derivative or fragment thereof.

Once the gene, derivative or fragment thereof has been introduced into a cell, the cell is cultured over sufficient time for the cells to acquire totipotency and proliferate. For example, culturing can continue at cell density of 1-100 thousand, for example, about 50 thousand per dish for cell culture.

The enhanced potency cells or reprogrammed totipotent cells may be obtained, for example, by culturing for 12 hours or longer, for example 1 day or longer, by using suitable medium for preparing totipotent or pluripotent cells, for example, medium for embryonic stem cells (for example, medium for human ES cells). The method described herein may require continuous culturing for 2 days or longer, for example 5 days or longer, 7 days or longer, and 10 days or longer.

In one embodiment, the method further comprises the step of selecting one or more cells which overexpress the TET family gene, derivative or fragment thereof.

In one embodiment, the one or more cells are selected using a marker gene.

In one embodiment, the marker gene can be selected from a drug resistance gene, a fluorescent protein gene, a chromogenic enzyme gene or a combination thereof. In a further embodiment, the marker gene is a drug resistance gene or a fluorescent protein gene.

Examples of drug resistance genes may include: a puromycin resistance gene, an ampicillin resistance gene, a neomycin resistance gene, a tetracycline resistance gene, a kanamycin resistance gene or a chloramphenicol resistance gene. Cells can be cultured on a medium containing the appropriate drug (i.e. a selection medium) and only those cells which incorporate and express the drug resistance gene will survive. Therefore, by culturing cells using a selection medium, it is possible to easily select cells comprising a drug resistance gene.

Examples of fluorescent protein genes include: a green fluorescent protein (GFP) gene, yellow fluorescent protein (YFP) gene, red fluorescent protein (RFP) gene or aequorin gene. Cells expressing the fluorescent protein gene can be detected using a fluorescence microscope and be selected using a cell sorter, such as a flow cytometer. Fluorescence-activated cell sorting (FACS) is a specialised type of flow cytometry that can be used to select the cells expressing the fluorescent protein.

In one embodiment, the one or more cells are selected using flow cytometry.

Examples of chromogenic enzyme genes include: β-galactosidase gene, β-glucuronidase gene, alkaline phosphatase gene, or secreted alkaline phosphatase SEAP gene. Cells expressing these chromogenic enzyme genes can be detected by applying the appropriate chromogenic substrate (e.g. X-gal for β-galatosidase) so that cells expressing the marker gene will produce a detectable colour (e.g. blue in a blue-white screen test).

All of the marker genes described herein are well known to those skilled in the art. For example, vectors containing such marker genes are commercially available from Invitrogen, Inc. (e.g. Gateway® Cloning Technology), Amersham Biosciences, Inc. and Promega, Inc.

According to a further aspect of the invention, there is provided a cell with enhanced potency obtainable by the method as defined herein.

According to a further aspect of the invention, there is provided a reprogrammed totipotent cell obtainable by the method as defined herein.

According to a further aspect of the invention, there is provided a nucleic acid comprising a TET3 isoform of SEQ ID NO: 11 or 13.

According to a further aspect of the invention, there is provided a vector comprising the nucleic acid as defined herein.

According to a further aspect of the invention, there is provided the use of the nucleic acid as defined herein, or the vector as defined herein, in a method of enhancing the potency of a cell.

According to a further aspect of the invention, there is provided the use of the nucleic acid as defined herein, or the vector as defined herein, in a method of reprogramming a cell to a totipotent state.

The enhanced potency cells or reprogrammed totipotent cells of the present invention have multiple uses in, for example, medical, chemical and agricultural industries.

The enhanced potency cells or reprogrammed totipotent cells of the present invention can be used in therapeutics, such as in cell or tissue regeneration. Human ES and iPS cells do not display markers of naïve pluripotency, therefore their utility in cell replacement therapy and as models of disease is limited. The present invention is able to move pluripotent cells into a higher level of potency which is able to overcome this issue.

The enhanced potency cells or reprogrammed totipotent cells of the present invention can be used in the generation of livestock and in large animal models. Current methods for cloning and genetic manipulation in large animals rely on somatic cell nuclear transfer (SCNT) technologies which can be restricted by poor self-renewal capability of modified cells. The development of ES and iPS cells in large animal models suffers from the same lack of potency observed in human ES and iPS cells (as described above). The present invention provides the generation of truly pluripotent or totipotent cells that are crucially able to proliferate and be manipulated in culture, thus streamlining genetic modification in livestock and in large animal models of disease. ‘Large animals’ include animals such as dogs, pigs, sheep, goats, cows and horses.

The enhanced potency cells or reprogrammed totipotent cells of the present invention can be used in methods of drug screening. For example, the cells could be differentiated into somatic cells, tissues or organs of interest, in order to test compounds or medicaments which could administered to the differentiated cells to assess their physiological activity or toxicity.

According to a further aspect of the invention, there is provided the cell with enhanced potency as defined herein for use in therapy.

According to a further aspect of the invention, there is provided the reprogrammed totipotent cell as defined herein for use in therapy.

In one embodiment, the therapy comprises tissue regeneration.

References herein to ‘tissue regeneration’ refer to therapies which restore the function of diseased and damaged organs and tissues by re-creating lost or damaged tissues.

Stem cells have the ability to develop into multiple types of tissue, therefore these cells can be introduced into damaged tissue in order to treat disease or injury. Examples of diseases or injuries in which enhanced potency cells or reprogrammed totipotent cells of the present invention may be used to treat include: anaemia, autoimmune diseases (e.g. arthritis, inflammatory bowel disease, Crohn's disease, diabetes, multiple sclerosis), birth defects, blindness, cancer, cardiovascular diseases (e.g. congestive heart failure, myocardial infarction, stroke), cirrhosis, deafness, degenerative disorders (e.g. Parkinson's disease), genetic disorders, Graft versus Host disease, immunodeficiency, infertility, ischaemia, lysosomal storage diseases, muscle damage (e.g. heart damage), neuronal damage (e.g. brain damage, spinal cord injury), neurodegenerative diseases (e.g. Alzheimer's disease, dementia, Huntingdon's disease), vision impairment and wound healing.

According to a further aspect of the invention, there is provided a kit comprising a vector containing a TET family gene, derivative or fragment thereof and instructions to use said kit in accordance with the method defined herein.

The kit may include one or more articles and/or reagents for performance of the method. For example, a TET family gene, derivative or fragment thereof, an oligonucleotide probe and/or pair of amplification primers for use in the methods described herein may be provided in isolated form and may be part of a kit, e.g. in a suitable container such as a vial in which the contents are protected from the external environment. The kit may include instructions for use of the nucleic acid, e.g. in PCR. A kit wherein the nucleic acid is intended for use in PCR may include one or more other reagents required for the reaction, such as polymerase, nucleotides, buffer solution etc.

In one embodiment, the kit additionally comprises at least one pluripotent cell. In an alternative embodiment, the kit additionally comprises at least one somatic cell.

In one embodiment, the kit additionally comprises a medium for culturing the cell and instructions for preparing the enhanced potency cells or reprogrammed totipotent cells in accordance with the method defined herein.

According to a further aspect of the invention, there is provided a method of reprogramming a cell to a pluripotent state, wherein said method comprises the step of introducing a TET3 gene, derivative or fragment thereof into the cell. In one embodiment the cell is a somatic cell.

It will be understood that this method may comprise the same method steps as defined herein for reprogramming a cell to a totipotent state. The introduction of TET3 into a cell results in a change in potency, e.g. to a pluripotent state. Therefore, introduction of TET3 into somatic cells leads to enhanced production of induced pluripotent stem cells.

The following studies illustrate the invention:

### EXAMPLE 1

### Identification of Tet3 Transcriptional Variants

An initial annotation of the Tet3 gene structure was provided by RefSeq (Accession No.: NM_183138). However, the presence of a large open reading frame upstream from this annotation indicated it was likely incomplete. 5′ amplification of cDNA ends was performed in ES cells and somatic tissues using the GeneRacer kit (Invitrogen) with primers specific to coding exons 1 and 3 (Table 1). This analysis identified two promoters, designated ‘Canonical’ and ‘Downstream’.

TABLE 1
Primers designed for 5′ amplification for
cDNA ends.

| | | SEQ ID |
|---|---|---|
| Primer | Sequence | No. |
| RACE Forward 1 | AACCCACTCACACCAACCCTCAG | 1 |
| RACE Forward 2 | CTGGACACACCGGCCAAGAAG | 2 |
| RACE Reverse 1 | AAGCCTGGGAGGTGGAATGAGAAG | 3 |
| RACE Reverse 2 | GGGCTCTCTAGCACCATTGACC | 4 |
| RACE Reverse 3 | GCCCTGCGGGAAATCATAAAG | 5 |

Examination of high-throughput RNA sequencing (RNA-seq) data from oocytes (Smallwood et al. (2011) Nat. Genet. 43, p. 811-814), ES cells (Cloonan et al., 2008) and multiple somatic tissues (Cloonan et al. (2008) Nat. Methods 5, p. 613-619; ESTs from GenBank) suggested the presence of an additional upstream promoter whose usage appeared restricted to oocytes (designated ‘Oocyte’).

The up-stream promoter may provide a mechanism for the oocyte and thus the zygote to accumulate high levels of TET3, and then switch to much lower levels of production in other tissues. In addition, within the oocyte-specific exon there is a predicted translational start site that is in-frame with the rest of the TET3 protein. This small peptide may play some role in modulating the function of TET3 in the oocyte. The RNA-seq data also indicates that transcripts produced in oocytes predominantly lack the first exon of the Tet3 gene, which encodes a CXXC domain. This domain possesses homologues in other epigenetic modifiers, such as DNA cytosine-5-methyltransferase 1 (DNMT1) and methyl-CpG binding domain protein 1 (MBD1), which are important for targeting the protein through binding to CpG islands. Recent studies suggest that the TET1 CXXC domain is capable of binding 5-methylcytosine (5mC) and 5-hydroxymethylcytosine (5hmC) in addition to unmethylated cytosine. Thus, differential incorporation of this domain may result in functional variation in the TET3 protein between oocyte and other tissues. It is also noteworthy that transcripts produced from the ‘Downstream’ promoter will lack the CXXC-encoding exon, permitting protein variation in cells other than oocytes.

### EXAMPLE 2

### Analysis of Tissue-Specific Transcript Variation

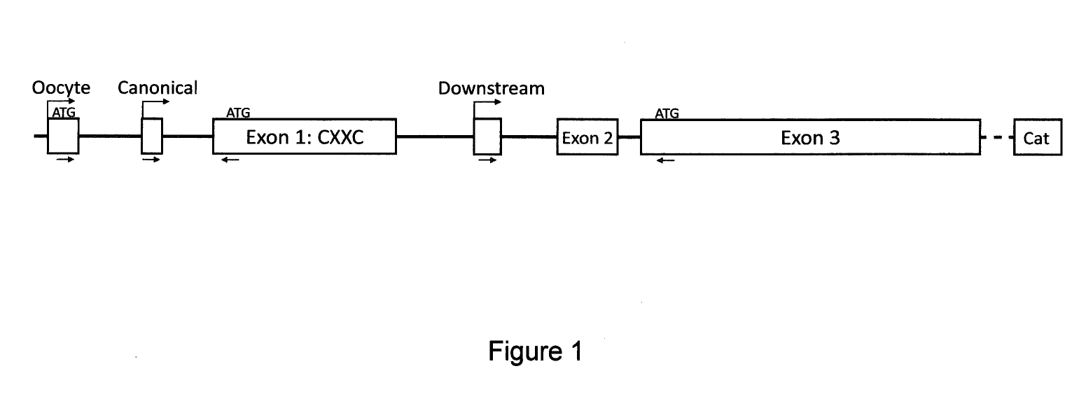

To confirm the specificity of the putative oocyte promoter and investigate the inclusion of the CXXC-encoding exon 1 in different cell types, primers were designed between each of the three promoters and either exon 1 or exon 3 (see Table 2) as indicated in FIG. 1. In effect, the former captures transcripts containing the CXXC-encoding exon, while the latter captures transcripts that lack this exon. These are therefore referred to as the CXXC(+) or CXXC(−) variants, respectively, of each promoter, with the exception of the Downstream promoter which can only produce CXXC(−) variants.

TABLE 2
Primers designed for promoter analysis

| | | SEQ |
|---|---|---|
| Primer | Sequence | ID No. |
| Oocyte Forward | GGGGTCGCACATGTTCCTC | 6 |
| Canonical Forward | GAAACTTTGCCCCTTTGTGC | 7 |
| Downstream Forward | CTCGGCGGGGATAATGG | 8 |
| Exon 1 Reverse | CTTGGCTGGGTGGGTTCT | 9 |
| Exon 3 Reverse | GCTTAGCTGCCTTGAATCTCCA | 10 |

RNA was extracted from E14 embryoid bodies, E14 ES cells, cortex, cerebellum, lung and spleen using Trizol (Invitrogen) and DNase treated with the DNA-free Kit (Ambion). cDNA was prepared with the SuperScriptIII First Strand Synthesis System (Invitrogen) using oligo (dT) primers.

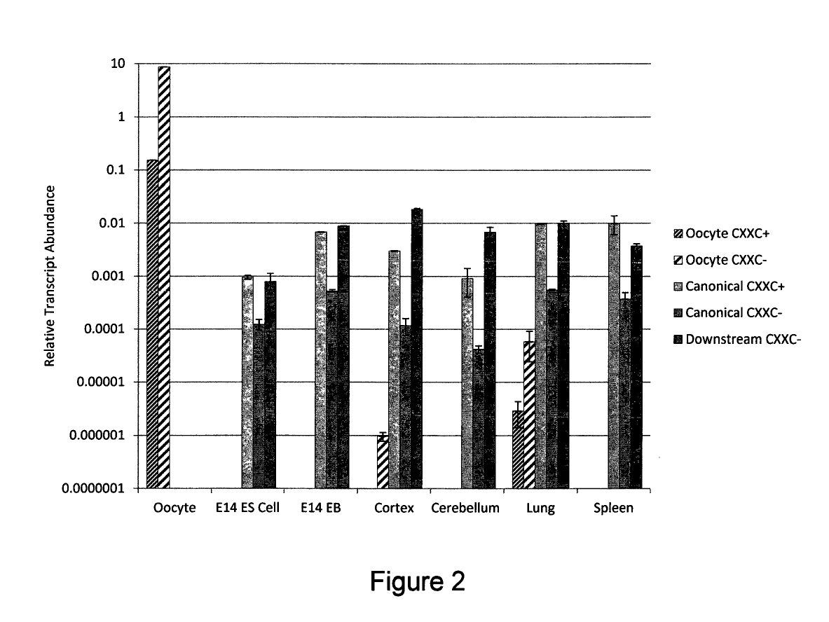

Quantitative PCR was performed using the Brilliant II SYBR Green qPCR Master Mix reagents (Agilent) on a Stratagene Mx3005P real-time system (Agilent). The Cₜ values of technical replicates were examined to ensure a discrepancy of less than 0.5 cycles. These replicates were then averaged and normalised against the average of two reference genes, Atp5b and Hspcb, using the ΔCₜ method (PfaffI (2004) Real Time PCR, p. 63-82). The results are summarised in FIG. 2.

This data confirms that meaningful usage of the oocyte promoter is restricted to oocytes amongst the tissues examined, and further demonstrates that oocytes employ exclusively this promoter. This indicates that the high expression of TET3 observed in oocytes is a function of promoter usage.

In addition, over 98% of TET3 transcripts in the oocyte lack the CXXC-encoding exon. This is consistent with bioinformatic analysis showing that splicing of the oocyte exon to exon 1 results in a truncated protein. In contrast, other cell types produce transcripts both with and without the CXXC-encoding exon using the canonical and downstream promoters. Thus TET3 protein present in oocytes and therefore zygotes contains a unique coding sequence and additionally contrasts with other examined tissues in the almost complete lack of CXXC exon inclusion. These transcriptional features may be linked to the specific role of TET3 in totipotent cells.

In summary, the data presented herein identifies the three major transcriptional variants produced from the Tet3 locus (see Table 3).

TABLE 3
Summary of Tet3 variants identified

| Variant | SEQ ID No. |
|---|---|
| Variant 1: Oocyte CXXC(−) | 11 |
| Variant 2: Canonical CXXC(−) | 12 |
| Variant 3: Canonical CXXC(+) | 13 |

### EXAMPLE 3

### Cloning and Overexpression of Tet3 Variants in ES Cells

Tet3 variant sequences were cloned into an inducible overexpression vector via several intermediary vectors using the Gateway system (Invitrogen). An overexpression vector was used which was designed to allow genomic incorporation using the piggyBAC system (Ding et al. (2005) Cell 122, p. 473-483; Wilson et al. (2007) Mol. Ther. 15, p. 139-145) that additionally contained an IRES-EGFP 3′ to the cloned sequence, hereafter referred to as pBAC.

Given its restriction to totipotent cells, Variant 1 (SEQ ID NO: 11) was chosen for initial overexpression analysis.

E14 ES cells were cultured in DMEM (with L-Glutamine, 4500 mg/L D-Glucose, 110 mg/L Sodium Pyruvate; Gibco) supplemented with 15% FBS (Fetal Bovine Serum, ES cell tested, Invitrogen), 1× MEM non-essential amino acids (Gibco), 1× Penicillin-Streptomycin (Gibco), 0.05 mM B-mercaptoethanol (1:1000, Gibco) and 10³ units/ml LIF (Leukemia Inhibitory Factor, ESGRO, Millipore) in 0.1% gelatin-coated plates, at 37° C. in humidified atmosphere with 5% CO₂. Media was changed daily and cells were split as indicated on reaching subconfluence, except when under selection.

FuGENE 6.0 (Roche) was used to transfect 1×10⁶ E14 ES cells with 2 μg each of pBAC construct and the other components of the piggyBAC system: a plasmid encoding the piggyBAC transposase and puromycin-selectable rtTA transactivator. The day after transfection, selection was applied through the addition of 1 μg/mL puromycin the medium and maintained thereafter.

The day before collection of cells, 1 μg/mL doxycycline was added to culture media to induce simultaneous expression of TET3 and green fluorescent protein (GFP). Cells were trypsinised and filtered then sorted into separate GFP positive (GFP+) and GFP negative (GFP−) populations using standard flow cytometry techniques.

### EXAMPLE 4

### Preliminary Gene Expression Analysis

RNA was extracted from sorted cells using DNA/RNA AllPrep Micro Kit (Qiagen), and DNase treated using the DNA-free Kit (Ambion). cDNA was prepared from 1 μg RNA using the SuperScript III First Strand Synthesis System (Invitrogen).

Previous work has shown that a small population of ES cells (referred to as ‘2-cell ES cells’) up-regulates genes associated with zygotic genome activation at the totipotent two-cell embryo stage, and display hallmarks of totipotency such as the ability to contribute to the extra-embryonic lineage (Macfarlan et al. (2012) Nature 487, p. 57-63). Given expression of TET3 is largely restricted to the oocyte and zygote and is present as a unique isoform at this stage, it was hypothesised that TET3 overexpression in ES cells would expand or enhance this population. Therefore the following candidates were selected based on their observed up-regulation at the two-cell stage and in 2-cell ES cells (Macfarlan et al. (2012) Nature 487, p. 57-63): MuERV-L, Zscan4c, Fgf5, Tbx3, Fbxo15, Pramel7, Mbd5, Calcoco2, Gm4340, Zfp352, Sp110, Tdpoz2, Tcstv3.

In addition, several genes expressed in ES cells but not predicted to be up-regulated were selected as controls: Tet1, Tcl1, Ooep.

Tet3 transcripts were also examined to verify its overexpression.

Primers for each of these genes were designed for quantitative RT-PCR, spanning intron-exon boundaries where possible (see Table 4).

TABLE 4
Summary of gene expression analysis primers

| | | SEQ |
|---|---|---|
| Primer | Sequence | ID No. |
| Candidate genes | | |
| Tet3 Forward | GGTCACAGCCTGCATGGACT | 14 |
| Tet3 Reverse | AGCGATTGTCTTCCTTGGTCAG | 15 |
| MuERVL pol | ATCTCCTGGCACCTGGTATG | 16 |
| Forward | | |
| MuERVL pol | AGAAGAAGGCATTTGCCAGA | 17 |
| Reverse | | |
| Zfp352 Forward | GGTTCACACATCCATCCCTACA | 18 |
| Zfp352 Reverse | CCTGGCTGGGAAGCACCT | 19 |
| Fgf5 Forward | GGGATTGTAGGAATACGAGGAGTTT | 20 |
| Fgf5 Reverse | TCTTGGCTTTCCCTCTCTTGTT | 21 |
| Gm4340 Forward | GGACGAAGTTTAGGGACAGCA | 22 |
| Gm4340 Reverse | TCCAGAGCCAGGGTTTCTTG | 23 |
| Sp110 Forward | CAGAATGAGGCAGGAGATTGG | 24 |
| Sp110 Reverse | AGCACATATCAGGTCAGGAGTTCA | 25 |
| Zscan4c | GAAACAACAGCAATCTGCAACAA | 26 |
| Forward | | |
| Zscan4c | TTCATTTCCACTACAGCTTTCACC | 27 |
| Reverse | | |
| Tdpoz2 Forward | ACACTCTCATCGTGGCTGACCT | 28 |
| Tdpoz2 Reverse | CAGGGAGCGGAATCTTTCATC | 29 |
| Tbx3 Forward | TCCACCTCCAACAACACGTTC | 30 |
| Tbx3 Reverse | AACTGCTGCTATCCGGCACT | 31 |
| Mbd5 Forward | CGCATCCTTCTCTGGTGCTC | 32 |
| Mbd5 Reverse | AGGTCTTGCATGTATAGCCTTCC | 33 |
| Tcstv3 Forward | GAATCTTGGACTTTACTTCCTCTCC | 34 |
| Tcstv3 Reverse | GTGGCTTTGCTCTTTGCTGA | 35 |
| Fbxo15 Forward | GCCTTGAATGGAGAACTGACTGT | 36 |
| Fbxo15 Reverse | AGCACACTGGAGAACTCACATACC | 37 |
| Pramel7 | CGGCATCTCACTATTGATGATGTC | 38 |
| Forward | | |
| Pramel7 | CTGACTGAGAGAGCTGGCACAG | 39 |
| Reverse | | |
| Calcoco2 | GCAAGGACTGGATTGGCATC | 40 |
| Forward | | |
| Calcoco2 | CTGCTGTGTGGCTGAATCCTT | 41 |
| Reverse | | |
| Control genes | | |
| Tet1 Forward | CCATTCTCACAAGGACATTCACA | 42 |
| Tet1 Reverse | GCAGGACGTGGAGTTGTTCA | 43 |
| Ooep Forward | CCACACGGCTGATGCTGA | 44 |
| Ooep Reverse | CTAGGTTCCCAGAGTTGACGG | 45 |
| Tcl1 Forward | CTCCATGTATTGGCAGATCCTGTA | 46 |
| Tcl1 Reverse | CTCCGAGTCTATCAGTTCAAGCAA | 47 |

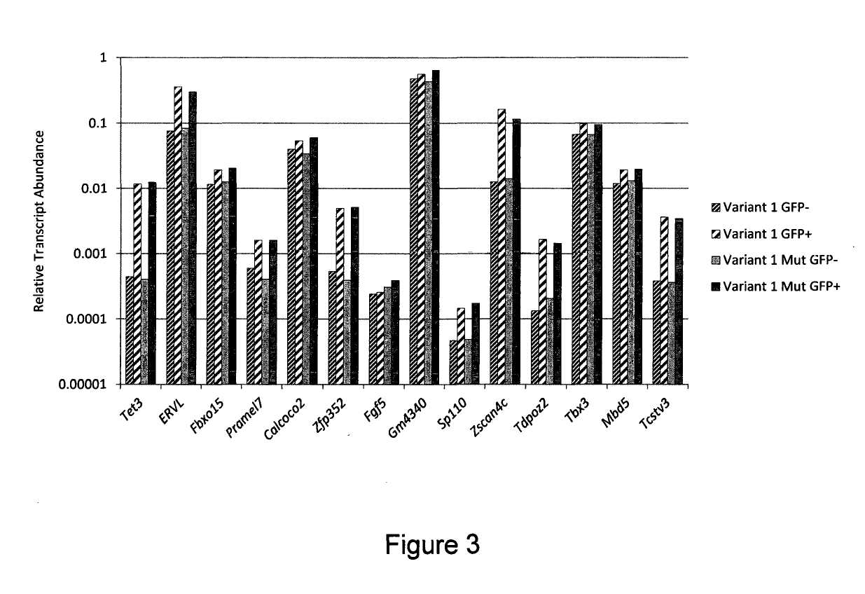

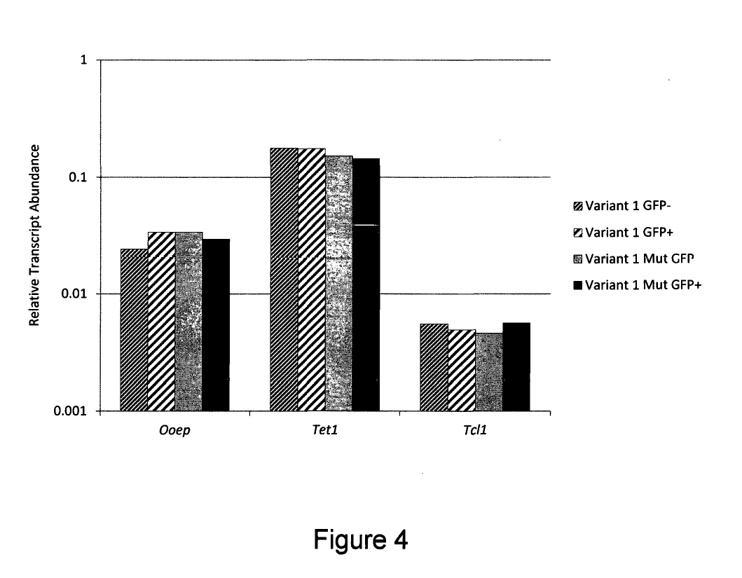

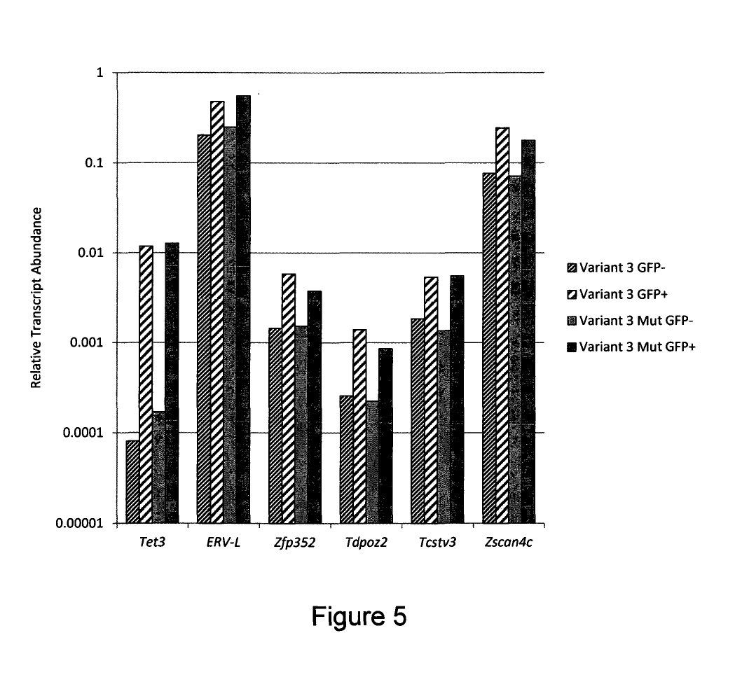

Quantitative PCR was performed using the Brilliant II SYBR Green qPCR Master Mix reagents (Agilent) on a C1000 Touch CFX384 Real Time System (BioRad). The Cₜ values of technical replicates were examined to ensure a discrepancy of less than 0.5 cycles. These replicates were then averaged and normalised against the average of two reference genes, Atp5b and Hspcb, using the ΔCₜ method (PfaffI (2004) Real-time PCR, p. 63-82). The results are summarised for Tet3 Variant 1 in FIG. 3 (candidate genes) and FIG. 4 (control genes) and for Tet3 Variant 3 in FIG. 5 (candidate genes).

Tet3 is up-regulated in the GFP positive cells as desired. Strikingly, all examined candidate genes show increased expression in cells expressing Tet3 Variant 1 and its catalytically inactive counterpart—including several whose expression is up-regulated approximately 10-fold—while control genes remain relatively stable. It is possible that large expression changes are occurring in a subpopulation of cells and are diluted by this global expression analysis, rather than a more modest up-regulation across the entire population. In either case, this data supports a shift towards to transcriptional program of the totipotent 2-cell stage which results in enhanced potency of TET3-overexpressing cells.

### EXAMPLE 5

### Genome-Wide Gene Expression Analysis by mRNA-Seq

Messenger RNA was isolated from 2 μg total RNA using Dynabeads mRNA Purification Kit (Invitrogen) and fragmented with RNA Fragmentation Reagent (Ambion). First strand cDNA synthesis was done with SuperScript III First Strand Synthesis System and 3 μgμl⁻¹ random hexamers (Invitrogen) followed by second strand synthesis with DNA Polymerase I and RNase H. After purification, a sequencing library was generated from the double stranded cDNA using paired-end adaptors (Illumina) with a Sanger index on PE2.0 and the NEBNext DNA Library Prep Master Mix Set for Illumina (NEB). Samples were sequenced with a single-end 50 bp protocol on one lane of an Illumina Hi-Seq 2000; the number of sequencing reads obtained for each indexed sample is given in Table 5. Messenger RNA-Seq data was mapped to the mouse genome (assembly NCBIM37) using TopHat (v1.4.1, options-g 1) in conjunction with gene models from Ensembl release 61.

TABLE 5
Read counts for mRNA-seq datasets

| Sample | Reads |
|---|---|
| Variant 1 GFP− | 52955484 |
| Variant 1 GFP+ | 47627618 |
| Variant 1 Mut GFP− | 57632592 |
| Variant 1 Mut GFP+ | 45503316 |

In a preliminary analysis, candidate genes that showed the largest upregulation in the qPCR data described above were examined for upregulation together with several members of their gene families: Pramel3, Pramel5, Pramel7, Sp110, Tdpoz1, Tdpoz3, Tdpoz4, Tdpoz5, Tet3, Zfp352, Zscan4c, Zscan4d, Zscan4e, Zscan4f and Zscan4-ps2.

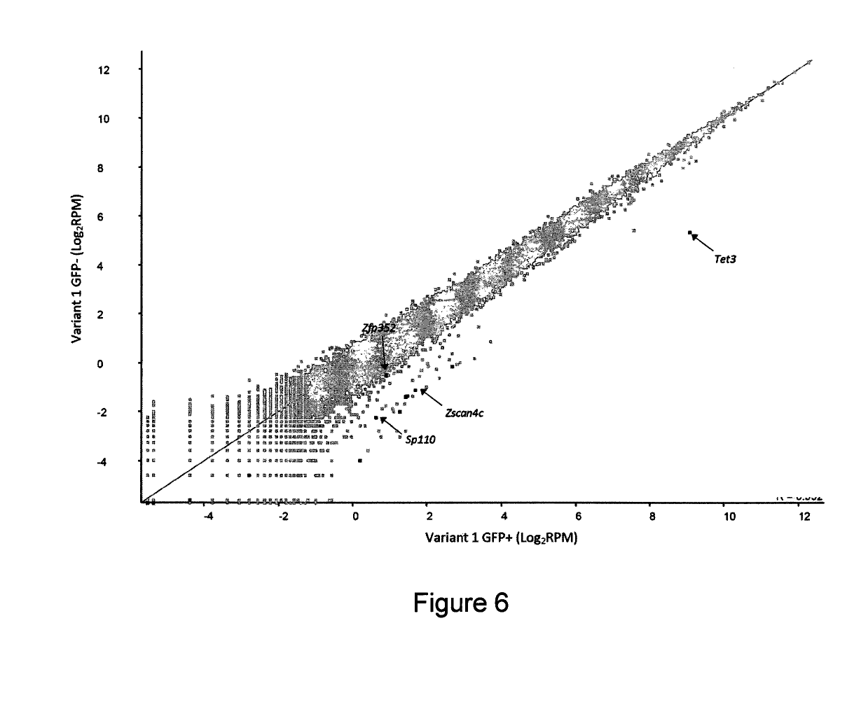

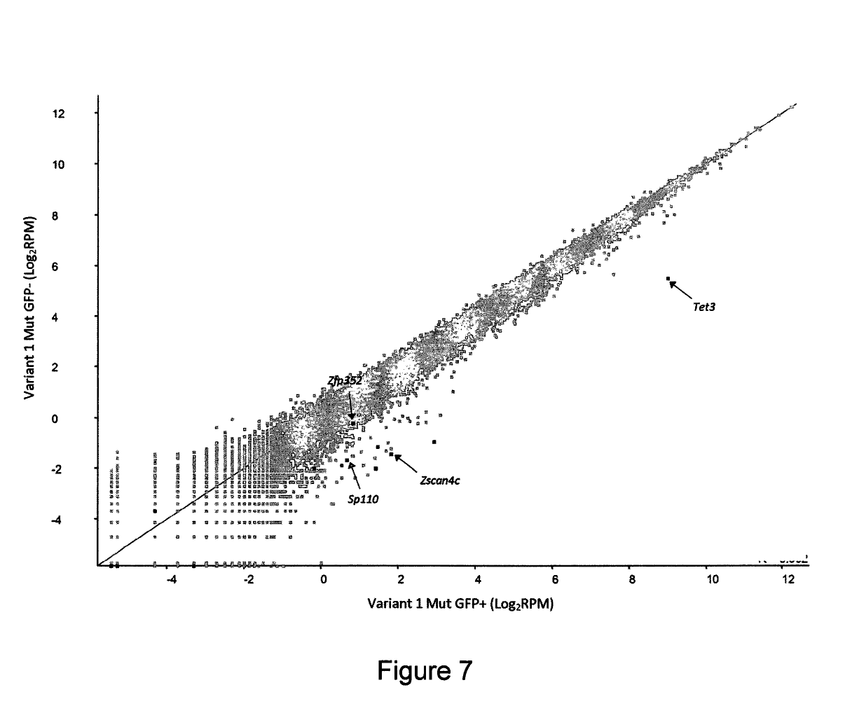

GFP positive and negative cells were compared on a scatterplot and the gene list above highlighted using SeqMonk v0.23.1 (FIGS. 6 and 7). Again, Tet3 is strongly up-regulated in GFP positive cells as expected. Strikingly, this analysis indicates that candidate genes and their family members are among the most up-regulated genes identified by unbiased genome-wide sequencing. Consistent with the qPCR data, overexpression of Tet3 Variant 1 or its catalytically inactivated counterpart have similar effects on gene expression, indicating that oxidase function is not required for the shift to a more ‘totipotent-like’ transcriptional programme.

### EXAMPLE 6

### Analysis of Totipotent-Like Subpopulation

Embryonic stem cell cultures are heterogeneous with respect to gene expression and developmental potency. They can be grouped into subpopulations characterised by expression of different marker genes. As individual cells cycle through different expression patterns, they move between different subpopulations. The abundance of a subpopulation is relatively stable within the same embryonic stem cell culture. In wildtype ES cells, a very small proportion of cells (5%) displays an expression profile characteristic of very early pre-implantation embryos. It is thought that these cells have an expanded potency phenotype compared to the vast majority of ES cells, and that they are responsible for the extremely rare cases in which ES cells contribute to extra-embryonic lineages in aggregations experiments.

The abundance of the totipotent-like subpopulation in ES cells expressing Tet3 Variant 1 was assessed. cDNA from individual GFP− and GFP+ cells was isolated using the C1 system (Fluidigm) with SMARTer cDNA amplification (Clontech). Steady state expression levels were analysed with the Biomark HD microfluidics system (Fluidigm) using EvaGreen qPCR chemistry (Bio-Rad). The following genes were used as markers for the totipotent-like subpopulation (highlighted in bold in Table 6): Zscan4c, MuERV-L, Arg2, Dub2a, Tcstv3, Lgals4.

Primers for each of these genes were designed for quantitative RT-PCR, spanning intron-exon boundaries where possible (see Table 6).

TABLE 6
Summary of single cell gene expression analysis primers

| Primer | Sequence | SEQ ID No. |
|---|---|---|
| Mervl_polnew_F | CCAACAGCAGAAACCAACACT | 48 |
| Mervl_polnew_R | AAGGCAAATCCATAACCAGAATA | 49 |
| Arg2_F | CTGGATCAAACCTTGCCTCTC | 50 |
| Arg2_R | ATCCCAAGTCGATCAATCTCTCTC | 51 |
| Dub2a_F | AATGCCTATGTGCTCTTCTATGTG | 52 |
| Dub2a_R | AGGTTTCTTTGGTTGCTTTCTTCT | 53 |
| Tcstv3_F | GAATCTTGGACTTTACTTCCTCTCC | 34 (see Table 4) |
| Tcstv3_R | GTGGCTTTGCTCTTTGCTGA | 35 (see Table 4) |
| Lgals4_F | CAGCTTTATGAATGGCTCTTGG | 54 |
| Lgals4_R | ATCTGGACGTAGGACAAGGTGA | 55 |
| Stat3_F | CGAGAGCAGCAAAGAAGGAG | 56 |
| Stat3_R | GGGTAGAGGTAGACAAGTGGAGAC | 57 |
| Serpine2_F | TTCCTTTCTTCATCTTGACCACA | 58 |
| Serpine2_R | ATCTTCTTCAGCACTTTACCAACTC | 59 |
| Stella_F | ATGAAGGACCCTGAAACTCCTC | 60 |
| Stella_R | ACTCTTGTTCTCCACAGGTACGG | 61 |
| Krt8_F | GACATCGAGATCACCACCTACC | 62 |
| Krt8_R | TTTCAATCTTCTTCACAACCACAG | 63 |
| Esrrb_F | GTATGCTATGCCTCCCAACGA | 64 |
| Esrrb_R | TACACGATGCCCAAGATGAGA | 65 |
| Tet2_F | GCCATTCTCAGGAGTCACTGC | 66 |
| Tet2_R | ACTTCTCGATTGTCTTCTCTATTGAGG | 67 |
| Ascl2_F | AGCCCGATGGAGCAGGAG | 68 |
| Ascl2_R | CCGAGCAGAGGTCAGTCAGC | 69 |
| Gata3_F | TCTGGAGGAGGAACGCTAATG | 70 |
| Gata3_R | GAGAGATGTGGCTCAGGGATG | 71 |
| Gata4_F | AGCAGCAGCAGTGAAGAGATG | 72 |
| Gata4_R | CGATGTCTGAGTGACAGGAGATG | 73 |
| Abcb5_F | GGTAGCACACAGGCTCTCCAC | 74 |
| Abcb5_R | ATGTCCTTGATTCCATTTGTTCAT | 75 |
| Tgfb2_F | CCTTCGCCCTCTTTACATTGAT | 76 |
| Tgfb2_R | GCTTCGGGATTTATGGTGTTG | 77 |
| Tdrd7_F | CCAATAGCAGGTTCAGTCCAAAG | 78 |
| Tdrd7_R | TAAGAGGCAGGAGGCGTGATA | 79 |
| Gata6_F | TCTACACAAGCGACCACCTCA | 80 |
| Gata6_R | GCCAGAGCACACCAAGAATC | 81 |
| Zfp352_F | GGTTCACACATCCATCCCTACA | 18 (see Table 4) |
| Zfp352_R | CCTGGCTGGGAAGCACCT | 19 (see Table 4) |
| Eomes_F | CACTGGATGAGGCAGGAGATTT | 82 |
| Eomes_R | GAGAAGGTGAAGGTCTGAGTCTTG | 83 |
| Brachyury_F | ATAACGCCAGCCCACCTACT | 84 |
| Brachyury_R | TCATACATCGGAGAACCAGAAGAC | 85 |
| Sox2_F | CAGCTCGCAGACCTACATGAAC | 86 |
| Sox2_R | CTGGAGTGGGAGGAAGAGGTAA | 87 |
| Tet1_F | CCATTCTCACAAGGACATTCACA | 42 (see Table 4) |
| Tet1_R | GCAGGACGTGGAGTTGTTCA | 43 (see Table 4) |
| Oct4_F | GCTGCTGAAGCAGAAGAGGAT | 88 |
| Oct4_R | TCCTGAAGGTTCTCATTGTTGTC | 89 |
| Nanog_F | TACCTCAGCCTCCAGCAGATG | 90 |
| Nanog_R | CCAGATGCGTTCACCAGATAG | 91 |
| Atp5b_F | GGCCAAGATGTCCTGCTGTT | 92 |
| Atp5b_R | GCTGGTAGCCTACAGCAGAAGG | 93 |
| Hsp90_F | GCTGGCTGAGGACAAGGAGA | 94 |
| Hsp90_R | CGTCGGTTAGTGGAATCTTCA | 95 |

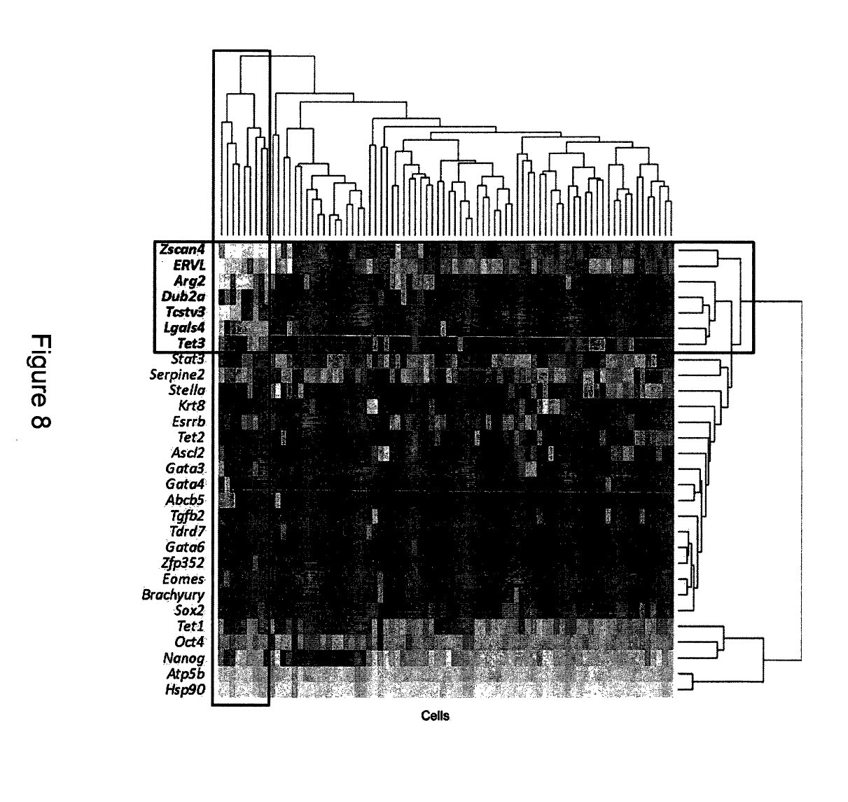

The single cell expression data was analysed using the SINGuLAR Analysis Toolset 2.0 (Fluidigm) and results of unsupervised clustering are shown as a heatmap with lighter colours representing higher expression (FIG. 8). Genes are clustered in a horizontal direction. Marker genes for a totipotent-like state are closely related and are highlighted in bold. Individual cells are clustered in a vertical direction. A subpopulation of closely related cells shows very high expression levels of totipotent-like marker genes (highlighted by a horizontal box) and was therefore designated ‘totipotent-like’ subpopulation. The proportion of cells falling in this category rises dramatically upon expression of Tet3 Variant 1. While in cells with no or very low expression of TET3 only 5% of cells are part of this subpopulation, in TET3 expressing cells the proportion increases to 40% (FIG. 9). Therefore, the shift towards a totipotent-like expression profile observed across the population is mediated by a dramatic expansion of the totipotent-like subpopulation.

### EXAMPLE 7

### Demonstration of Enhanced Potency by Transdifferentiation Assay

ES cells are pluripotent as they can generate the many different cell-types of the embryo, but not extra-embryonic tissues such as the trophoblast. The ability to form trophoblast-like cells in growth conditions used for trophoblast stem (TS) cell culture thus provides an in vitro assay of expanded potency (Ng et al. (2008) Nat Cell Biol. 10, 1280-1290). This test was applied to wild-type E14 ES cells and two ES cell lines constitutively overexpressing Tet3 variant 1 (referred to as Tet3 clone 2 and Tet3 clone 7). As positive controls, genetically modified cell lines either overexpressing a Ras transgene (referred to as iRas) or lacking Oct4 expression (referred to as ZHBTc4) that are known to undergo significant transdifferentiation were tested in parallel (Niwa et al. (2000) Nat. Genet. 24, 372-376; Niwa et al. (2005) Cell 123, 917-929).

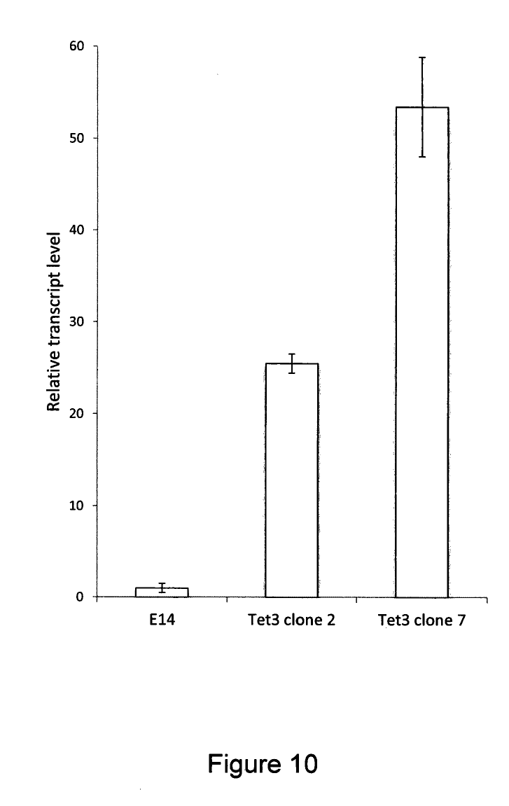

In order to link any observed changes to levels of TET3 expression, qRT-PCR analysis was performed on wild-type E14 ES cells and the two Tet3-overexpressing ES cell lines as previously described hereinbefore (FIG. 10). Tet3 clone 7 expresses TET3 approximately 2-fold more than Tet3 clone 2; both these cell lines have markedly increased Tet3 transcript levels relative to E14 cells.

Transdifferentiation Assays

TS base media consisting of RPMI 1640 supplemented with 20% FBS, 1 mM sodium pyruvate, 50 U/mL penicillin-streptomycin and 0.05 mM B-mercaptoethanol was conditioned by incubation with irradiated mouse embryonic fibroblast (MEF) cells on cell culture dishes for two days and passed through a 0.22 μm filter. Complete TS cell medium was prepared by combining 70% conditioned media, 30% TS base media, 20 ng/mL β-foetal growth factor and 1 μg/mL heparin.

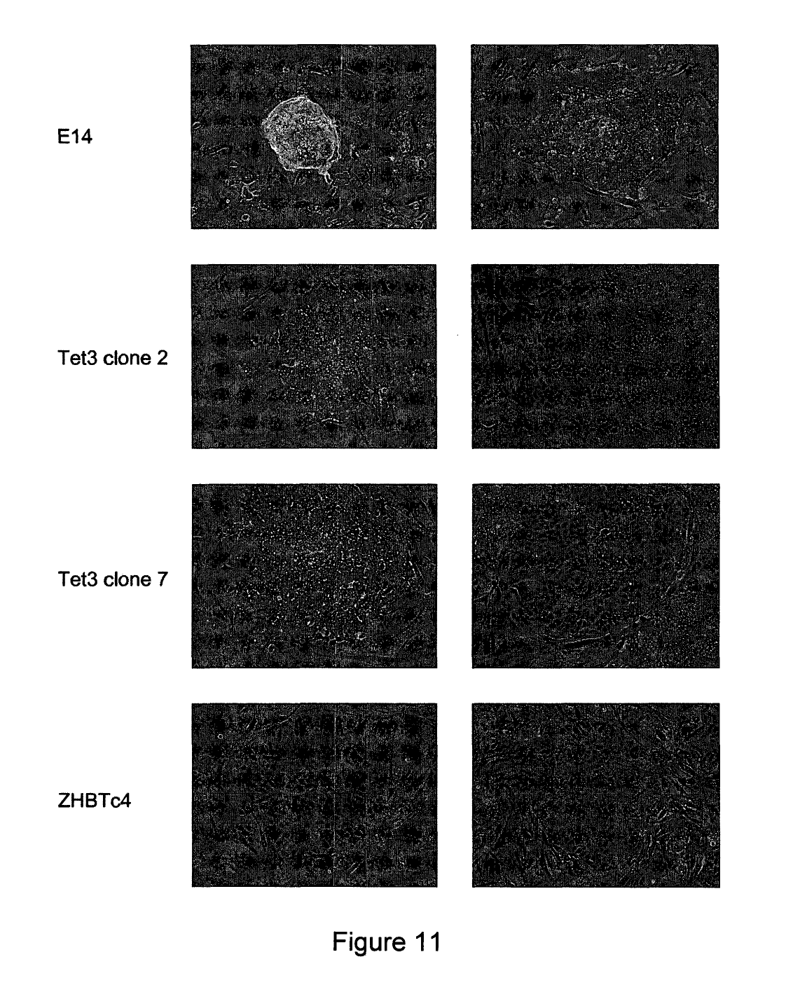

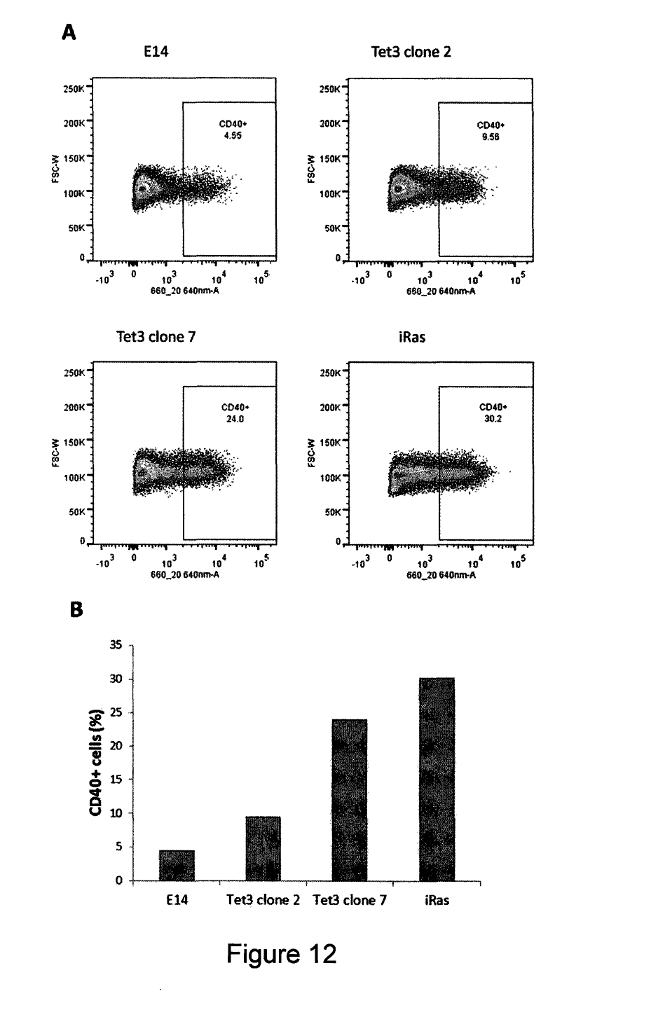

After six days of culture in complete TS cell medium, transdifferentiation was assessed by morphology (FIG. 11) and flow cytometry analysis of the TS cell marker CD40 (FIG. 12).

Examination of representative phase-contrast images reveals a significant shift towards the trophoblast-like morphology of ZHBTc4 cells in TET3-overexpressing cell lines that was largely absent in E14 cells. This effect was more pronounced in the Tet3 clone 7 cell line.

CD40 is an established marker for discrimination of TS and ES cells (Rugg-Gunn et al. (2012) Cell 22, 887-901). Flow cytometry analysis demonstrates a clear increase in the number of CD40-positive cells upon TET3 overexpression. Statistically testing of the entire cell population confirms a highly significant change for both TET3-overexpressing cell lines relative to E14 ES cells (Student's t test; p<0.0001 in both cases). Again, the change is more extensive in the Tet3 clone 7 cell line, reaching a level of CD40-positive cells almost equal that observed in the positive control iRas cell line.

This data shows that overexpression of TET3 in ES cells results in a strong enhancement of the ability to transdifferentiate to a trophoblast-like state, demonstrating a gain in developmental potency. Furthermore, this expansion of potency is linked to the dose of TET3 received by the cells; in both analyses, the cell line with higher TET3 expression (clone 7) showed a greater effect.

1. A method of enhancing the potency of a cell, wherein said method comprises the step of introducing a TET family gene, derivative or fragment thereof into the cell.

2. The method according to claim 1, wherein the cell is enhanced to a totipotent state.

3. The method according to claim 1, wherein the cell is enhanced to a pluripotent state, such as a true pluripotent state.

4. The method according to any one of claims 1 to 3, wherein the TET family gene, derivative or fragment thereof, is TET2 or TET3.

5. The method according to any one of claims 1 to 4, wherein the TET family gene, derivative or fragment thereof, is TET3.

6. The method according to any one of claims 1 to 5, wherein the TET family gene, derivative or fragment thereof, is a TET3 isoform of SEQ ID NO: 11 or 13.

7. The method according to any one of claims 1, 2 or 4 to 6, wherein the cell is a pluripotent cell, such as an embryonic stem (ES) cell, in particular an E14 embryonic stem (ES) cell.

8. The method according to any one of claims 1 to 6, wherein the cell is a somatic cell.

9. The method according to any one of claims 1 to 8, wherein the introducing step comprises transfecting the cell with a vector containing the TET family gene, derivative or fragment thereof.

10. The method according to claim 9, wherein the vector is a transposon vector.

11. A method of preparing a cell with enhanced potency which comprises the step of introducing a TET family gene, derivative or fragment thereof into a cell.

12. The method according to claim 11, therein the cell is a pluripotent cell, such as an embryonic stem (ES) cell, in particular an E14 embryonic stem (ES) cell.

13. The method according to claim 11, wherein the cell is a somatic cell.

14. The method according to claim 13, further comprising the step of introducing a Oct3/4 gene, a Sox2 gene, a Klf4 gene and a c-Myc gene into the somatic cell.

15. The method according to any one of claims 11 to 14, further comprising the step of culturing the cell after introduction of the TET family gene, derivative or fragment thereof.

16. The method according to any one of claims 11 to 15, further comprising the step of selecting one or more cells which overexpress the TET family gene, derivative or fragment thereof.

17. The method according to claim 16, wherein the one or more cells are selected using flow cytometry.

18. A cell with enhanced potency obtainable by the method defined in any one of claims 1 to 17.

19. A nucleic acid comprising a TET3 isoform of SEQ ID NO: 11 or 13.

20. A vector comprising the nucleic acid according to claim 19.

21. Use of the nucleic acid according to claim 19, or the vector according to claim 20 in a method of enhancing the potency of a cell.

22. The cell with enhanced potency according to claim 18 for use in therapy.

23. The cell with enhanced potency for use according to claim 22, wherein the therapy comprises tissue regeneration.

24. A kit comprising a vector containing a TET family gene, derivative or fragment thereof and instructions to use said kit in accordance with the method defined in any one of claims 1 to 17.

25. The kit according to claim 24, additionally comprising at least one pluripotent cell, such as an embryonic stem (ES) cell, in particular an E14 embryonic stem (ES) cell.

26. The kit according to claim 24, additionally comprising at least one somatic cell.

27. The kit according to claim 25 or claim 26, additionally comprising a medium for culturing the cell and instructions for preparing the cells with enhanced potency in accordance with the method defined in any one of claims 1 to 17.


SEQUENCE LISTING (95 sequences)

SEQ ID NO: 1 - DNA, 23 bp, Artificial
  Synthetic primer
aacccactcacaccaaccctcag

SEQ ID NO: 2 - DNA, 21 bp, Artificial
  Synthetic primer
ctggacacaccggccaagaag

SEQ ID NO: 3 - DNA, 24 bp, Artificial
  Synthetic primer
aagcctgggaggtggaatgagaag

SEQ ID NO: 4 - DNA, 22 bp, Artificial
  Synthetic primer
gggctctctagcaccattgacc

SEQ ID NO: 5 - DNA, 21 bp, Artificial
  Synthetic primer
gccctgcgggaaatcataaag

SEQ ID NO: 6 - DNA, 19 bp, Artificial
  Synthetic primer
ggggtcgcacatgttcctc

SEQ ID NO: 7 - DNA, 20 bp, Artificial
  Synthetic primer
gaaactttgcccctttgtgc

SEQ ID NO: 8 - DNA, 17 bp, Artificial
  Synthetic primer
ctcggcggggataatgg

SEQ ID NO: 9 - DNA, 18 bp, Artificial
  Synthetic primer
cttggctgggtgggttct

SEQ ID NO: 10 - DNA, 22 bp, Artificial
  Synthetic primer
gcttagctgccttgaatctcca

SEQ ID NO: 11 - DNA, 5142 bp, Mus musculus
atgttcctcccagaaacccctcaacaatatgctgtggaaataaatgctcgtgaaggaacggggccctgggcacaaggggcgactgtcaagacaggctcagagctcagcccagttgatggacctgttccaggtcagatggactcagggccagtgtaccatggagattcaaggcagctaagcacctcaggggcgccggtcaatggtgctagagagcccgccggacccggtcttctgggagctgcgggtccttggcgggtagaccagaagcccgactgggaggctgcctcaggccccactcacgctgctcgtctggaagatgcccacgacctggtggccttttcggccgtggccgaagctgtgtcatcttacggggcccttagtacccggctctatgaaaccttcaaccgtgagatgagtcgtgaggctgggagcaacggcaggggcccccggcctgagagctgctctgagggcagtgaagacctggacacgctgcagacagccctggcccttgcaaggcatggcatgaaaccacccaactgcacctgcgatggcccagagtgccccgacttcctcgagtggctggagggcaagatcaagtctatggccatggagggagggcaggggcggcctaggctcccaggcgctctgcctcccagtgaggctggcctcccagcccctagcaccagaccgccactccttagctctgaggtcccccaggtacctcccctggagggcctgcctctgtcccagagcgcgctgagcattgccaaggaaaaaaacatcagcctgcagacagccatcgccatcgaggccctcacacagctctcctccgccctccctcagccttctcattccacctcccaggcttcttgtccactccctgaggccttgtccccttctgcccctttcaggtctccccagtcctacctccgggccccctcatggcctgtggttcccccagaggaacatccatcctttgctcctgacagcccagccttccctccagcaaccccaagacctgagttttctgaagcgtggggcactgacacccccccagcgacaccccggaactcctggcctgtacctcgcccaagccctgaccctatggcagaactggagcagctattgggcagcgccagtgattacatccagtcagtattcaagcggcctgaggccctgcccaccaagcccaaggtcaaggttgaggccccctcttcttcccctgctccggtaccatctcctatttctcagagggaggctcccctgctgtcttcagagcctgacacccaccagaaggcccagacagcccttcagcaacatcttcatcacaagcgcaacctattcttggaacaggcccaagatgcctccttccctacttccacagagcctcaggctcctggttggtgggcccctcccggctcacctgccccaaggcctcctgacaaaccacccaaggaaaagaaaaagaagccccccacccctgctggaggtcccgtgggagcagagaaaaccacccctgggatcaagaccagtgtccgaaagcccattcagatcaagaaatccaggtccagggacatgcagcccctcttcctgcctgttaggcagattgttctggaagggctaaaaccccaagcctcagaaggacaggcaccgttacccgcccagctctctgtcccacctcctgcctcccagggtgctgcatcccagagctgtgccacccctctaaccccagaaccttctcttgcgctatttgcacctagtccctccggggacagcctgctgccccctactcaggaaatgagatcccccagccccatggtagccctgcagtcaggctccactggtggcccccttccccctgccgatgacaagctggaggagctcatccggcaatttgaggctgaatttggggatagctttgggcttcccggcccaccttcggtgcccattcaagaacctgaaaaccaatcaacatgtctcccagctccggagagcccttttgccacccgctcccccaagaagatcaagatcgagtcctcaggggccgtgactgtgctctcaactacctgcttccattcagaagaggggggacaggaggccacgcccaccaaggctgagaacccactcacaccaaccctcagtggcttcttggagtcacctctaaagtacctagacacacctactaagagtctgctggacacaccggccaagaaggctcagtccgagttccctacctgcgattgtgtcgaacaaatagtggagaaagatgaaggcccatattacactcacctgggatctggccccacagtagcttctatccgggaactcatggaggatcggtatggagaaaaggggaaagctatccggattgagaaggtcatctacacgggcaaggaggggaagagttctcgaggctgtcccatcgccaagtgggtgatccgaagacacacactggaggagaagctgctgtgcctggtgcggcatcgggcaggccaccattgtcagaacgccgtgattgttatcttgatcctggcctgggagggcatccctcgaagccttggggacaccctctaccaggagcttactgataccctccggaagtatggcaaccctaccagccggagatgtggcctcaatgatgaccggacctgtgcttgccaaggcaaagaccctaacacctgcggtgcctccttctccttcggctgttcctggagcatgtacttcaacggctgcaaatatgctcggagcaagacgccacgaaagttccgcctcacgggagacaatccgaaggaggaggaggtgctccggaatagctttcaggatctggccactgaagttgctcccctctacaagcggctcgcaccccaggcctatcagaaccaggtgaccaatgaggatgtggcgatcgactgccgcctggggctgaaggaagggagacccttctcaggggtcacagcctgcatggacttctgtgcccacgcccacaaggaccaacataacctctacaatgggtgcactgtggtctgcaccctgaccaaggaagacaatcgctgcgtgggccagatccctgaggacgagcaactgcacgtgctgcccctctacaagatggccagcacggatgagtttggcagcgaggaaaaccagaacgccaaggtcagtagtggggccatccaggtgctcacagcattccccagagaggtccggcggctgcctgagcctgccaagtcctgccgccaacggcagctggaagccaggaaggcggcggccgagaagaagaagctgcagaaggagaaactgagcacgccagagaagatcaagcaggaggccctggagttggctggagtcaccactgacccaggcctgtctctgaagggtggattgtcccagcaaagcctgaagccctccctcaaggtggagcctcagaaccactttagctcctttaagtacagtggcaatgcggtggtggaaagctactcggtgctgggcagctgccggccctccgacccctacagcatgagcagtgtgtattcctaccattcgcgctatgcacagcctggcctggcctctgtcaacggcttccactccaagtacacacttccctcctttggctactatggctttccatcaagcaaccctgtcttcccctcccagttcctgggtcccagtgcctgggggcatgggggcagtggaggcagttttgagaagaagccagacctccatgctctacacaacagcctgaacccagcctacggtggtgctgagtttgccgagctgccaggtcaggctgttgccacagacaaccaccaccccatccctcaccaccagcagcctgcttacccaggccccaaggaatatctgctacccaaggtcccccagctccacccagcatccagggacccctctccctttgctcagagttccagttgctacaacagatccatcaagcaagagccaatagaccctctgacccaggctgagtccattcccagagactctgctaagatgagtagaacacccttgccggaagcatctcagaatgggggacccagtcatctgtggggacagtactcaggaggcccaagcatgtccccgaagaggactaacagtgtaggtggcaactggggcgtgttccctccgggggagagccctaccattgttcccgacaagctcaattcttttggggccagctgtctcactccttcacacttcccagaaagccagtggggactgttcactggtgaaggccagcagtcggccccccatgctggagcacggcttcgaggcaagccatggagcccctgcaagtttgggaacggcacctctgccttgactggtcccagcctaactgagaagccatgggggatgggaaccggggatttcaaccccgccctgaaaggtggacctgggttccaagacaagttgtggaatcctgtgaaggtggaggagggcaggattcccacaccgggggccaacccgctagacaaagcctggcaagcctttggcatgcccttgagctccaacgagaagctatttggggccctgaagtcagaggagaaactgtgggatcccttcagcctggaggaggggacagctgaggagccccccagcaagggggtggtgaaggaagagaagagtggacccacagtggaagaggacgaggaggaactgtggtcggacagtgaacacaacttcctggatgagaacataggcggggtggccgtggcccccgcccattgctccatcctcatcgagtgtgcccggcgagagctgcatgccaccactccactcaaaaaacccaaccgctgccaccccacccgcatctcgctggtcttctaccaacacaagaacctcaaccagcccaaccacgggctggcgctctgggaggccaagatgaagcagctggcggaacgggcgcggcagcggcaagaggaggccgcacgcctgggcctgggccagcaggaggccaagctctacgggaagaagcgaaaatgggggggtgctatggtggctgagccccagcacaaagaaaagaagggggctatccctacccggcaggcgctggccatgcccacagactccgcggtcaccgtgtcctcttacgcctacacaaaggtcactggcccctacagccgctggatctag

SEQ ID NO: 12 - DNA, 5007 bp, Mus musculus
atggactcagggccagtgtaccatggagattcaaggcagctaagcacctcaggggcgccggtcaatggtgctagagagcccgccggacccggtcttctgggagctgcgggtccttggcgggtagaccagaagcccgactgggaggctgcctcaggccccactcacgctgctcgtctggaagatgcccacgacctggtggccttttcggccgtggccgaagctgtgtcatcttacggggcccttagtacccggctctatgaaaccttcaaccgtgagatgagtcgtgaggctgggagcaacggcaggggcccccggcctgagagctgctctgagggcagtgaagacctggacacgctgcagacagccctggcccttgcaaggcatggcatgaaaccacccaactgcacctgcgatggcccagagtgccccgacttcctcgagtggctggagggcaagatcaagtctatggccatggagggagggcaggggcggcctaggctcccaggcgctctgcctcccagtgaggctggcctcccagcccctagcaccagaccgccactccttagctctgaggtcccccaggtacctcccctggagggcctgcctctgtcccagagcgcgctgagcattgccaaggaaaaaaacatcagcctgcagacagccatcgccatcgaggccctcacacagctctcctccgccctccctcagccttctcattccacctcccaggcttcttgtccactccctgaggccttgtccccttctgcccctttcaggtctccccagtcctacctccgggccccctcatggcctgtggttcccccagaggaacatccatcctttgctcctgacagcccagccttccctccagcaaccccaagacctgagttttctgaagcgtggggcactgacacccccccagcgacaccccggaactcctggcctgtacctcgcccaagccctgaccctatggcagaactggagcagctattgggcagcgccagtgattacatccagtcagtattcaagcggcctgaggccctgcccaccaagcccaaggtcaaggttgaggccccctcttcttcccctgctccggtaccatctcctatttctcagagggaggctcccctgctgtcttcagagcctgacacccaccagaaggcccagacagcccttcagcaacatcttcatcacaagcgcaacctattcttggaacaggcccaagatgcctccttccctacttccacagagcctcaggctcctggttggtgggcccctcccggctcacctgccccaaggcctcctgacaaaccacccaaggaaaagaaaaagaagccccccacccctgctggaggtcccgtgggagcagagaaaaccacccctgggatcaagaccagtgtccgaaagcccattcagatcaagaaatccaggtccagggacatgcagcccctcttcctgcctgttaggcagattgttctggaagggctaaaaccccaagcctcagaaggacaggcaccgttacccgcccagctctctgtcccacctcctgcctcccagggtgctgcatcccagagctgtgccacccctctaaccccagaaccttctcttgcgctatttgcacctagtccctccggggacagcctgctgccccctactcaggaaatgagatcccccagccccatggtagccctgcagtcaggctccactggtggcccccttccccctgccgatgacaagctggaggagctcatccggcaatttgaggctgaatttggggatagctttgggcttcccggcccaccttcggtgcccattcaagaacctgaaaaccaatcaacatgtctcccagctccggagagcccttttgccacccgctcccccaagaagatcaagatcgagtcctcaggggccgtgactgtgctctcaactacctgcttccattcagaagaggggggacaggaggccacgcccaccaaggctgagaacccactcacaccaaccctcagtggcttcttggagtcacctctaaagtacctagacacacctactaagagtctgctggacacaccggccaagaaggctcagtccgagttccctacctgcgattgtgtcgaacaaatagtggagaaagatgaaggcccatattacactcacctgggatctggccccacagtagcttctatccgggaactcatggaggatcggtatggagaaaaggggaaagctatccggattgagaaggtcatctacacgggcaaggaggggaagagttctcgaggctgtcccatcgccaagtgggtgatccgaagacacacactggaggagaagctgctgtgcctggtgcggcatcgggcaggccaccattgtcagaacgccgtgattgttatcttgatcctggcctgggagggcatccctcgaagccttggggacaccctctaccaggagcttactgataccctccggaagtatggcaaccctaccagccggagatgtggcctcaatgatgaccggacctgtgcttgccaaggcaaagaccctaacacctgcggtgcctccttctccttcggctgttcctggagcatgtacttcaacggctgcaaatatgctcggagcaagacgccacgaaagttccgcctcacgggagacaatccgaaggaggaggaggtgctccggaatagctttcaggatctggccactgaagttgctcccctctacaagcggctcgcaccccaggcctatcagaaccaggtgaccaatgaggatgtggcgatcgactgccgcctggggctgaaggaagggagacccttctcaggggtcacagcctgcatggacttctgtgcccacgcccacaaggaccaacataacctctacaatgggtgcactgtggtctgcaccctgaccaaggaagacaatcgctgcgtgggccagatccctgaggacgagcaactgcacgtgctgcccctctacaagatggccagcacggatgagtttggcagcgaggaaaaccagaacgccaaggtcagtagtggggccatccaggtgctcacagcattccccagagaggtccggcggctgcctgagcctgccaagtcctgccgccaacggcagctggaagccaggaaggcggcggccgagaagaagaagctgcagaaggagaaactgagcacgccagagaagatcaagcaggaggccctggagttggctggagtcaccactgacccaggcctgtctctgaagggtggattgtcccagcaaagcctgaagccctccctcaaggtggagcctcagaaccactttagctcctttaagtacagtggcaatgcggtggtggaaagctactcggtgctgggcagctgccggccctccgacccctacagcatgagcagtgtgtattcctaccattcgcgctatgcacagcctggcctggcctctgtcaacggcttccactccaagtacacacttccctcctttggctactatggctttccatcaagcaaccctgtcttcccctcccagttcctgggtcccagtgcctgggggcatgggggcagtggaggcagttttgagaagaagccagacctccatgctctacacaacagcctgaacccagcctacggtggtgctgagtttgccgagctgccaggtcaggctgttgccacagacaaccaccaccccatccctcaccaccagcagcctgcttacccaggccccaaggaatatctgctacccaaggtcccccagctccacccagcatccagggacccctctccctttgctcagagttccagttgctacaacagatccatcaagcaagagccaatagaccctctgacccaggctgagtccattcccagagactctgctaagatgagtagaacacccttgccggaagcatctcagaatgggggacccagtcatctgtggggacagtactcaggaggcccaagcatgtccccgaagaggactaacagtgtaggtggcaactggggcgtgttccctccgggggagagccctaccattgttcccgacaagctcaattcttttggggccagctgtctcactccttcacacttcccagaaagccagtggggactgttcactggtgaaggccagcagtcggccccccatgctggagcacggcttcgaggcaagccatggagcccctgcaagtttgggaacggcacctctgccttgactggtcccagcctaactgagaagccatgggggatgggaaccggggatttcaaccccgccctgaaaggtggacctgggttccaagacaagttgtggaatcctgtgaaggtggaggagggcaggattcccacaccgggggccaacccgctagacaaagcctggcaagcctttggcatgcccttgagctccaacgagaagctatttggggccctgaagtcagaggagaaactgtgggatcccttcagcctggaggaggggacagctgaggagccccccagcaagggggtggtgaaggaagagaagagtggacccacagtggaagaggacgaggaggaactgtggtcggacagtgaacacaacttcctggatgagaacataggcggggtggccgtggcccccgcccattgctccatcctcatcgagtgtgcccggcgagagctgcatgccaccactccactcaaaaaacccaaccgctgccaccccacccgcatctcgctggtcttctaccaacacaagaacctcaaccagcccaaccacgggctggcgctctgggaggccaagatgaagcagctggcggaacgggcgcggcagcggcaagaggaggccgcacgcctgggcctgggccagcaggaggccaagctctacgggaagaagcgaaaatgggggggtgctatggtggctgagccccagcacaaagaaaagaagggggctatccctacccggcaggcgctggccatgcccacagactccgcggtcaccgtgtcctcttacgcctacacaaaggtcactggcccctacagccgctggatctag

SEQ ID NO: 13 - DNA, 5412 bp, Mus musculus
atgagccagtttcaggtgcccttggcggtccagccggacctgtcaggactttatgatttcccgcagggccaggtgatggtagggggcttccaggggcctgggcttcctatggctgggagtgagacccaactgcgagggggtggagatgggcggaagaaaaggaaacggtgtgggacctgcgatccctgccgacggctggaaaactgtgggtcttgtaccagctgcaccaatcgtcgcacacaccagatctgcaaactccgcaagtgtgaggtgctgaagaaaaaagcggggcttcttaaggaggtggaaataaatgctcgtgaaggaacggggccctgggcacaaggggcgactgtcaagacaggctcagagctcagcccagttgatggacctgttccaggtcagatggactcagggccagtgtaccatggagattcaaggcagctaagcacctcaggggcgccggtcaatggtgctagagagcccgccggacccggtcttctgggagctgcgggtccttggcgggtagaccagaagcccgactgggaggctgcctcaggccccactcacgctgctcgtctggaagatgcccacgacctggtggccttttcggccgtggccgaagctgtgtcatcttacggggcccttagtacccggctctatgaaaccttcaaccgtgagatgagtcgtgaggctgggagcaacggcaggggcccccggcctgagagctgctctgagggcagtgaagacctggacacgctgcagacagccctggcccttgcaaggcatggcatgaaaccacccaactgcacctgcgatggcccagagtgccccgacttcctcgagtggctggagggcaagatcaagtctatggccatggagggagggcaggggcggcctaggctcccaggcgctctgcctcccagtgaggctggcctcccagcccctagcaccagaccgccactccttagctctgaggtcccccaggtacctcccctggagggcctgcctctgtcccagagcgcgctgagcattgccaaggaaaaaaacatcagcctgcagacagccatcgccatcgaggccctcacacagctctcctccgccctccctcagccttctcattccacctcccaggcttcttgtccactccctgaggccttgtccccttctgcccctttcaggtctccccagtcctacctccgggccccctcatggcctgtggttcccccagaggaacatccatcctttgctcctgacagcccagccttccctccagcaaccccaagacctgagttttctgaagcgtggggcactgacacccccccagcgacaccccggaactcctggcctgtacctcgcccaagccctgaccctatggcagaactggagcagctattgggcagcgccagtgattacatccagtcagtattcaagcggcctgaggccctgcccaccaagcccaaggtcaaggttgaggccccctcttcttcccctgctccggtaccatctcctatttctcagagggaggctcccctgctgtcttcagagcctgacacccaccagaaggcccagacagcccttcagcaacatcttcatcacaagcgcaacctattcttggaacaggcccaagatgcctccttccctacttccacagagcctcaggctcctggttggtgggcccctcccggctcacctgccccaaggcctcctgacaaaccacccaaggaaaagaaaaagaagccccccacccctgctggaggtcccgtgggagcagagaaaaccacccctgggatcaagaccagtgtccgaaagcccattcagatcaagaaatccaggtccagggacatgcagcccctcttcctgcctgttaggcagattgttctggaagggctaaaaccccaagcctcagaaggacaggcaccgttacccgcccagctctctgtcccacctcctgcctcccagggtgctgcatcccagagctgtgccacccctctaaccccagaaccttctcttgcgctatttgcacctagtccctccggggacagcctgctgccccctactcaggaaatgagatcccccagccccatggtagccctgcagtcaggctccactggtggcccccttccccctgccgatgacaagctggaggagctcatccggcaatttgaggctgaatttggggatagctttgggcttcccggcccaccttcggtgcccattcaagaacctgaaaaccaatcaacatgtctcccagctccggagagcccttttgccacccgctcccccaagaagatcaagatcgagtcctcaggggccgtgactgtgctctcaactacctgcttccattcagaagaggggggacaggaggccacgcccaccaaggctgagaacccactcacaccaaccctcagtggcttcttggagtcacctctaaagtacctagacacacctactaagagtctgctggacacaccggccaagaaggctcagtccgagttccctacctgcgattgtgtcgaacaaatagtggagaaagatgaaggcccatattacactcacctgggatctggccccacagtagcttctatccgggaactcatggaggatcggtatggagaaaaggggaaagctatccggattgagaaggtcatctacacgggcaaggaggggaagagttctcgaggctgtcccatcgccaagtgggtgatccgaagacacacactggaggagaagctgctgtgcctggtgcggcatcgggcaggccaccattgtcagaacgccgtgattgttatcttgatcctggcctgggagggcatccctcgaagccttggggacaccctctaccaggagcttactgataccctccggaagtatggcaaccctaccagccggagatgtggcctcaatgatgaccggacctgtgcttgccaaggcaaagaccctaacacctgcggtgcctccttctccttcggctgttcctggagcatgtacttcaacggctgcaaatatgctcggagcaagacgccacgaaagttccgcctcacgggagacaatccgaaggaggaggaggtgctccggaatagctttcaggatctggccactgaagttgctcccctctacaagcggctcgcaccccaggcctatcagaaccaggtgaccaatgaggatgtggcgatcgactgccgcctggggctgaaggaagggagacccttctcaggggtcacagcctgcatggacttctgtgcccacgcccacaaggaccaacataacctctacaatgggtgcactgtggtctgcaccctgaccaaggaagacaatcgctgcgtgggccagatccctgaggacgagcaactgcacgtgctgcccctctacaagatggccagcacggatgagtttggcagcgaggaaaaccagaacgccaaggtcagtagtggggccatccaggtgctcacagcattccccagagaggtccggcggctgcctgagcctgccaagtcctgccgccaacggcagctggaagccaggaaggcggcggccgagaagaagaagctgcagaaggagaaactgagcacgccagagaagatcaagcaggaggccctggagttggctggagtcaccactgacccaggcctgtctctgaagggtggattgtcccagcaaagcctgaagccctccctcaaggtggagcctcagaaccactttagctcctttaagtacagtggcaatgcggtggtggaaagctactcggtgctgggcagctgccggccctccgacccctacagcatgagcagtgtgtattcctaccattcgcgctatgcacagcctggcctggcctctgtcaacggcttccactccaagtacacacttccctcctttggctactatggctttccatcaagcaaccctgtcttcccctcccagttcctgggtcccagtgcctgggggcatgggggcagtggaggcagttttgagaagaagccagacctccatgctctacacaacagcctgaacccagcctacggtggtgctgagtttgccgagctgccaggtcaggctgttgccacagacaaccaccaccccatccctcaccaccagcagcctgcttacccaggccccaaggaatatctgctacccaaggtcccccagctccacccagcatccagggacccctctccctttgctcagagttccagttgctacaacagatccatcaagcaagagccaatagaccctctgacccaggctgagtccattcccagagactctgctaagatgagtagaacacccttgccggaagcatctcagaatgggggacccagtcatctgtggggacagtactcaggaggcccaagcatgtccccgaagaggactaacagtgtaggtggcaactggggcgtgttccctccgggggagagccctaccattgttcccgacaagctcaattcttttggggccagctgtctcactccttcacacttcccagaaagccagtggggactgttcactggtgaaggccagcagtcggccccccatgctggagcacggcttcgaggcaagccatggagcccctgcaagtttgggaacggcacctctgccttgactggtcccagcctaactgagaagccatgggggatgggaaccggggatttcaaccccgccctgaaaggtggacctgggttccaagacaagttgtggaatcctgtgaaggtggaggagggcaggattcccacaccgggggccaacccgctagacaaagcctggcaagcctttggcatgcccttgagctccaacgagaagctatttggggccctgaagtcagaggagaaactgtgggatcccttcagcctggaggaggggacagctgaggagccccccagcaagggggtggtgaaggaagagaagagtggacccacagtggaagaggacgaggaggaactgtggtcggacagtgaacacaacttcctggatgagaacataggcggggtggccgtggcccccgcccattgctccatcctcatcgagtgtgcccggcgagagctgcatgccaccactccactcaaaaaacccaaccgctgccaccccacccgcatctcgctggtcttctaccaacacaagaacctcaaccagcccaaccacgggctggcgctctgggaggccaagatgaagcagctggcggaacgggcgcggcagcggcaagaggaggccgcacgcctgggcctgggccagcaggaggccaagctctacgggaagaagcgaaaatgggggggtgctatggtggctgagccccagcacaaagaaaagaagggggctatccctacccggcaggcgctggccatgcccacagactccgcggtcaccgtgtcctcttacgcctacacaaaggtcactggcccctacagccgctggatctag

SEQ ID NO: 14 - DNA, 20 bp, Artificial
  Synthetic primer
ggtcacagcctgcatggact

SEQ ID NO: 15 - DNA, 22 bp, Artificial
  Synthetic primer
agcgattgtcttccttggtcag

SEQ ID NO: 16 - DNA, 20 bp, Artificial
  Synthetic primer
atctcctggcacctggtatg

SEQ ID NO: 17 - DNA, 20 bp, Artificial
  Synthetic primer
agaagaaggcatttgccaga

SEQ ID NO: 18 - DNA, 22 bp, Artificial
  Synthetic primer
ggttcacacatccatccctaca

SEQ ID NO: 19 - DNA, 18 bp, Artificial
  Synthetic primer
cctggctgggaagcacct

SEQ ID NO: 20 - DNA, 25 bp, Artificial
  Synthetic primer
gggattgtaggaatacgaggagttt

SEQ ID NO: 21 - DNA, 22 bp, Artificial
  Synthetic primer
tcttggctttccctctcttgtt

SEQ ID NO: 22 - DNA, 21 bp, Artificial
  Synthetic primer
ggacgaagtttagggacagca

SEQ ID NO: 23 - DNA, 20 bp, Artificial
  Synthetic primer
tccagagccagggtttcttg

SEQ ID NO: 24 - DNA, 21 bp, Artificial
  Synthetic primer
cagaatgaggcaggagattgg

SEQ ID NO: 25 - DNA, 24 bp, Artificial
  Synthetic primer
agcacatatcaggtcaggagttca

SEQ ID NO: 26 - DNA, 23 bp, Artificial
  Synthetic primer
gaaacaacagcaatctgcaacaa

SEQ ID NO: 27 - DNA, 24 bp, Artificial
  Synthetic primer
ttcatttccactacagctttcacc

SEQ ID NO: 28 - DNA, 22 bp, Artificial
  Synthetic primer
acactctcatcgtggctgacct

SEQ ID NO: 29 - DNA, 21 bp, Artificial
  Synthetic primer
cagggagcggaatctttcatc

SEQ ID NO: 30 - DNA, 21 bp, Artificial
  Synthetic primer
tccacctccaacaacacgttc

SEQ ID NO: 31 - DNA, 20 bp, Artificial
  Synthetic primer
aactgctgctatccggcact

SEQ ID NO: 32 - DNA, 20 bp, Artificial
  Synthetic primer
cgcatccttctctggtgctc

SEQ ID NO: 33 - DNA, 23 bp, Artificial
  Synthetic primer
aggtcttgcatgtatagccttcc

SEQ ID NO: 34 - DNA, 25 bp, Artificial
  Synthetic primer
gaatcttggactttacttcctctcc

SEQ ID NO: 35 - DNA, 20 bp, Artificial
  Synthetic primer
gtggctttgctctttgctga

SEQ ID NO: 36 - DNA, 23 bp, Artificial
  Synthetic primer
gccttgaatggagaactgactgt

SEQ ID NO: 37 - DNA, 24 bp, Artificial
  Synthetic primer
agcacactggagaactcacatacc

SEQ ID NO: 38 - DNA, 24 bp, Artificial
  Synthetic primer
cggcatctcactattgatgatgtc

SEQ ID NO: 39 - DNA, 22 bp, Artificial
  Synthetic primer
ctgactgagagagctggcacag

SEQ ID NO: 40 - DNA, 20 bp, Artificial
  Synthetic primer
gcaaggactggattggcatc

SEQ ID NO: 41 - DNA, 21 bp, Artificial
  Synthetic primer
ctgctgtgtggctgaatcctt

SEQ ID NO: 42 - DNA, 23 bp, Artificial
  Synthetic primer
ccattctcacaaggacattcaca

SEQ ID NO: 43 - DNA, 20 bp, Artificial
  Synthetic primer
gcaggacgtggagttgttca

SEQ ID NO: 44 - DNA, 18 bp, Artificial
  Synthetic primer
ccacacggctgatgctga

SEQ ID NO: 45 - DNA, 21 bp, Artificial
  Synthetic primer
ctaggttcccagagttgacgg

SEQ ID NO: 46 - DNA, 24 bp, Artificial
  Synthetic primer
ctccatgtattggcagatcctgta

SEQ ID NO: 47 - DNA, 24 bp, Artificial
  Synthetic primer
ctccgagtctatcagttcaagcaa

SEQ ID NO: 48 - DNA, 21 bp, Artificial
  Synthetic primer
ccaacagcagaaaccaacact

SEQ ID NO: 49 - DNA, 23 bp, Artificial
  Synthetic primer
aaggcaaatccataaccagaata

SEQ ID NO: 50 - DNA, 21 bp, Artificial
  Synthetic primer
ctggatcaaaccttgcctctc

SEQ ID NO: 51 - DNA, 24 bp, Artificial
  Synthetic primer
atcccaagtcgatcaatctctctc

SEQ ID NO: 52 - DNA, 24 bp, Artificial
  Synthtetic primer
aatgcctatgtgctcttctatgtg

SEQ ID NO: 53 - DNA, 24 bp, Artificial
  Synthetic primer
aggtttctttggttgctttcttct

SEQ ID NO: 54 - DNA, 22 bp, Artificial
  Synthetic primer
cagctttatgaatggctcttgg

SEQ ID NO: 55 - DNA, 22 bp, Artificial
  Synthetic primer
atctggacgtaggacaaggtga

SEQ ID NO: 56 - DNA, 20 bp, Artificial
  Synthetic primer
cgagagcagcaaagaaggag

SEQ ID NO: 57 - DNA, 24 bp, Artificial
  Synthetic primer
gggtagaggtagacaagtggagac

SEQ ID NO: 58 - DNA, 23 bp, Artificial
  Synthetic primer
ttcctttcttcatcttgaccaca

SEQ ID NO: 59 - DNA, 25 bp, Artificial
  Synthetic primer
atcttcttcagcactttaccaactc

SEQ ID NO: 60 - DNA, 22 bp, Artificial
  Synthetic primer
atgaaggaccctgaaactcctc

SEQ ID NO: 61 - DNA, 23 bp, Artificial
  Synthetic primer
actcttgttctccacaggtacgg

SEQ ID NO: 62 - DNA, 22 bp, Artificial
  Synthetic primer
gacatcgagatcaccacctacc

SEQ ID NO: 63 - DNA, 24 bp, Artificial
  Synthetic primer
tttcaatcttcttcacaaccacag

SEQ ID NO: 64 - DNA, 21 bp, Artificial
  Synthetic primer
gtatgctatgcctcccaacga

SEQ ID NO: 65 - DNA, 21 bp, Artificial
  Synthetic primer
tacacgatgcccaagatgaga

SEQ ID NO: 66 - DNA, 21 bp, Artificial
  Synthetic primer
gccattctcaggagtcactgc

SEQ ID NO: 67 - DNA, 27 bp, Artificial
  Synthetic primer
acttctcgattgtcttctctattgagg

SEQ ID NO: 68 - DNA, 18 bp, Artificial
  Synthetic primer
agcccgatggagcaggag

SEQ ID NO: 69 - DNA, 20 bp, Artificial
  Synthetic primer
ccgagcagaggtcagtcagc

SEQ ID NO: 70 - DNA, 21 bp, Artificial
  Synthetic primer
tctggaggaggaacgctaatg

SEQ ID NO: 71 - DNA, 21 bp, Artificial
  Synthetic primer
gagagatgtggctcagggatg

SEQ ID NO: 72 - DNA, 21 bp, Artificial
  Synthetic primer
agcagcagcagtgaagagatg

SEQ ID NO: 73 - DNA, 23 bp, Artificial
  Synthetic primer
cgatgtctgagtgacaggagatg

SEQ ID NO: 74 - DNA, 21 bp, Artificial
  Synthetic primer
ggtagcacacaggctctccac

SEQ ID NO: 75 - DNA, 24 bp, Artificial
  Synthetic primer
atgtccttgattccatttgttcat

SEQ ID NO: 76 - DNA, 22 bp, Artificial
  Synthetic primer
ccttcgccctctttacattgat

SEQ ID NO: 77 - DNA, 21 bp, Artificial
  Synthetic primer
gcttcgggatttatggtgttg

SEQ ID NO: 78 - DNA, 23 bp, Artificial
  Synthetic primer
ccaatagcaggttcagtccaaag

SEQ ID NO: 79 - DNA, 21 bp, Artificial
  Synthetic primer
taagaggcaggaggcgtgata

SEQ ID NO: 80 - DNA, 21 bp, Artificial
  Synthetic primer
tctacacaagcgaccacctca

SEQ ID NO: 81 - DNA, 20 bp, Artificial
  Synthetic primer
gccagagcacaccaagaatc

SEQ ID NO: 82 - DNA, 22 bp, Artificial
  Synthetic primer
cactggatgaggcaggagattt

SEQ ID NO: 83 - DNA, 24 bp, Artificial
  Synthetic primer
gagaaggtgaaggtctgagtcttg

SEQ ID NO: 84 - DNA, 20 bp, Artificial
  Synthetic primer
ataacgccagcccacctact

SEQ ID NO: 85 - DNA, 24 bp, Artificial
  Synthetic primer
tcatacatcggagaaccagaagac

SEQ ID NO: 86 - DNA, 22 bp, Artificial
  Synthetic primer
cagctcgcagacctacatgaac

SEQ ID NO: 87 - DNA, 22 bp, Artificial
  Synthetic primer
ctggagtgggaggaagaggtaa

SEQ ID NO: 88 - DNA, 21 bp, Artificial
  Synthetic primer
gctgctgaagcagaagaggat

SEQ ID NO: 89 - DNA, 23 bp, Artificial
  Synthetic primer
tcctgaaggttctcattgttgtc

SEQ ID NO: 90 - DNA, 21 bp, Artificial
  Synthetic primer
tacctcagcctccagcagatg

SEQ ID NO: 91 - DNA, 21 bp, Artificial
  Synthetic primer
ccagatgcgttcaccagatag

SEQ ID NO: 92 - DNA, 20 bp, Artificial
  Synthetic primer
ggccaagatgtcctgctgtt

SEQ ID NO: 93 - DNA, 22 bp, Artificial
  Synthetic primer
gctggtagcctacagcagaagg

SEQ ID NO: 94 - DNA, 20 bp, Artificial
  Synthetic primer
gctggctgaggacaaggaga

SEQ ID NO: 95 - DNA, 21 bp, Artificial
  Synthetic primer
cgtcggttagtggaatcttca